(19) United States
(12) Patent Application Publication
(10) Pub. No.: US 2025/0051843 A1
(43) Pub. Date: Feb. 13, 2025

(54) METHODS FOR HUMAN LEUKOCYTE ANTIGEN TYPING AND PHASING

(71) Applicant: Dovetail Genomics, LLC, Scotts Valley, CA (US)

(72) Inventor: JAMES DURBIN, Scotts Valley, CA (US)

(21) Appl. No.: 18/780,961

(22) Filed: Jul. 23, 2024

Related U.S. Application Data
(63) Continuation of application No. PCT/US23/11549, filed on Jan. 25, 2023.
(60) Provisional application No. 63/302,812, filed on Jan. 25, 2022.

(51) Int. Cl. C12Q 1/6881; C12Q 1/6806; C12Q 1/6874; G16B 30/10

(52) U.S. Cl. CPC C12Q 1/6881; C12Q 1/6874; G16B 30/10; C12Q 1/6806

(57) ABSTRACT

Provided herein are methods of obtaining phased human leukocyte antigen (HLA) types of a sample from an individual.

### CROSS-REFERENCE

This application is a continuation of International Patent Application No. PCT/US23/11549, filed Jan. 25, 2023, which claims the benefit of U.S. Provisional Application No. 63/302,812, filed Jan. 25, 2022, which is incorporated herein by reference in its entirety.

### BACKGROUND

The human leukocyte antigen (HLA) system, also known as the major histocompatibility complex (MHC), is a complex of genes spanning approximately three megabases on chromosome 6 which encodes cell-surface proteins responsible for regulation of the immune system. HLAs present peptides from inside the cell to T-lymphocytes. These peptides are produced from digested proteins that are broken down in the proteosome. In general these peptides are small polymers of about 8-14 amino acids in length. HLAs corresponding to MHC class I (A, B, and C), all of which are the HLA class I group, present peptides to CD8+ cytotoxic T cells. HLAs corresponding to MHC class II (DP, DM, DO, DQ, and DR) present antigens to CD4+ helper T cells. HLA genes are highly polymorphic allowing HLAs to differentiate self cells from non-self cells. Any cell displaying some other HLA type is “non-self” and is seen as an invader by the body's immune system. Therefore, HLA typing is very important in organ transplants. High resolution HLA typing is needed in identifying a full match for transplant, even when the donor is related.

### SUMMARY

In an aspect, there are provided methods obtaining a phased human leukocyte antigen (HLA) type of a sample. In some cases, the method comprises aligning a plurality of sequencing reads to a reference genome, wherein at least a portion of said plurality of sequencing reads correspond to a HLA gene locus. In some cases, the method comprises identifying a plurality of single nucleotide polymorphisms (SNPs) and/or indels in said plurality of sequencing reads. In some cases, the method comprises sorting said plurality of SNPs and indels into phase blocks. In some cases, the method comprises aligning said plurality of sequencing reads to a variation graph to identify a plurality of HLA types. In some cases, the method comprises comparing said plurality of HLA types to a plurality of known HLA alleles to obtain a SNP signature for each HLA allele. In some cases, the method comprises comparing said SNP signature of (e) to said phase blocks of (c) to obtain said phased HLA type. Alternatively or in combination, the method comprises identifying said plurality of SNPs by comparing said plurality of sequencing reads to said variation graph. In some cases, said plurality of sequencing reads are obtained by cross-linking said sample, fragmenting nucleic acids in said sample to produce nucleic acid fragments, ligating said nucleic acid fragments to produce ligated nucleic acid fragments, reversing crosslinks, and sequencing said ligated nucleic acid fragments. In some cases, said sample is crosslinked by contacting said sample to a crosslinking agent selected from formaldehyde, psoralen, disuccinimidyl glutarate (DSG), ethylene glycol bis(succinimidyl succinate) (EGS), ultraviolet light, or a combination thereof. In some cases, said fragmenting comprising contacting said sample to an enzyme. In some cases, said enzyme is a nuclease, a restriction endonuclease, a transposase, or a combination thereof. In some cases, said nuclease is a micrococcal nuclease. In some cases, said transposase is Tn5. In some cases, fragmenting comprises non-enzymatic cleavage. In some cases, the method further comprises, subsequent to said ligating, adding a label said nucleic acid fragments. In some cases, said label comprises biotin. In some cases, said label comprises an oligonucleotide. In some cases, said oligonucleotide comprises a barcode. In some cases, said cross-linking links nucleic acids to nucleic acid binding proteins in said sample. In some cases, said plurality of SNPs and/or indels are identified by aligning said sequencing reads to said variation graph. In some cases, said phased HLA type comprises HLA-A, HLA-B, HLA-C, HLA-DRB1, HLA-DRB3, HLA-DRB4, HLA-DRB5, HLA-DQA1, HLA-DQB1, HLA-DPA1, and/or HLA-DPB1. In some cases, said sample comprises nucleic acids enriched for HLA sequences. In some cases, the entire HLA region is phased with over 90% of the phased SNPs in a single phase block. In some cases, major and minor HLA type groups are phased. In some cases, the method is completed in less than 30 hours of CPU time. In some cases, the method is completed in less than 15 hours, less than 10 hours, less than 5 hours, less than 4 hours, less than 3 hours, less than 2 hours, or less than 1 hour of CPU time. In some cases, the method is completed in about 30 to about 40 minutes of CPU time. In some cases, the sequencing reads comprise paired end reads. In some cases, the sequencing reads comprise long reads.

In another aspect, there are provided computer-implemented methods of obtaining a phased human leukocyte antigen (HLA) type of a sample. In some cases, the method comprises aligning a plurality of sequencing reads to a reference genome or a variation graph, wherein at least a portion of said plurality of sequencing reads correspond to a HLA gene locus. In some cases, the method comprises identifying a plurality of single nucleotide polymorphisms (SNPs) and/or indels in said plurality of sequencing reads. In some cases, the method comprises sorting said plurality of SNPs and indels into phase blocks. In some cases, the method comprises aligning said plurality of sequencing reads to said variation graph to identify a plurality of HLA types. In some cases, the method comprises comparing said plurality of HLA types to a plurality of known HLA alleles to obtain a SNP signature for each HLA allele. In some cases, the method comprises comparing said SNP signature to said phase blocks to obtain said phased HLA type. In some cases, the sequencing reads comprise paired end reads. In some cases, the sequencing reads comprise long reads.

In another aspect, there are provided methods of obtaining a phased human leukocyte antigen (HLA) type of a sample. In some cases, the method comprises aligning a plurality of sequencing reads to a variation graph, wherein at least a portion of said plurality of sequencing reads correspond to a HLA gene locus. In some cases, the method comprises identifying a plurality of single nucleotide polymorphisms (SNPs) and/or indels in said plurality of sequencing reads. In some cases, the method comprises sorting said plurality of SNPs and indels into phase blocks. In some cases, the method comprises aligning said plurality of sequencing reads to said variation graph to identify a plurality of HLA types. In some cases, the method comprises comparing said plurality of HLA types to a plurality of known HLA alleles to obtain a SNP signature for each HLA allele. In some cases, the method comprises comparing said SNP signature of (e) to said phase blocks of (c) to obtain said phased HLA type. In some cases, the sequencing reads comprise paired end reads. In some cases, the sequencing reads comprise long reads.

In another aspect, there are provided methods of obtaining a phased human leukocyte antigen (HLA) type of a sample. In some cases, the method comprises aligning a plurality of sequencing reads to a reference genome or a variation graph, wherein at least a portion of said plurality of sequencing reads correspond to a HLA gene locus. In some cases, the method comprises identifying a plurality of single nucleotide polymorphisms (SNPs) and/or indels in said plurality of sequencing reads. In some cases, the method comprises sorting said plurality of SNPs and indels into a plurality of phase blocks. In some cases, the method comprises comparing said plurality of SNPs with a plurality of SNP signatures of known HLA types to identify a plurality of HLA types. In some cases, the method comprises comparing said SNP signature to said phase blocks to obtain said phased HLA type. In some cases, comparing said SNP signature to said phase blocks comprises assigning a phase of a HLA type of said plurality of HLA types based on a quantity of SNPs of said plurality of SNPs that match with said plurality of SNP signatures. In some cases, sorting said plurality of SNPs and indels into a plurality of phase blocks comprises assigning a phase to each of said plurality of phase blocks. In some cases, the method further comprises generating a database comprising a plurality of SNP signatures of known HLA types. In some cases said generating said database comprises aligning a plurality of HLA alleles sequences with a reference genome. In some cases, said plurality of sequencing reads comprises reads generated using a proximity ligation technique. In some cases, said proximity ligation technique comprises Hi-C, Chicago, Micro-C, or Omni-C. In some cases, said plurality of sequencing reads comprises a contiguous sequence comprising sequences derived from regions of a chromosomes that are distal from one another in the natural sequence of the chromosome. In some cases, the method further comprises comprising prior to aligning a plurality of sequencing reads to a reference genome or variation graph, generating a plurality of sequencing reads by (i) subjecting said sample to a proximity ligation reaction, (ii) generating a sequencing library derived from nucleic acids in said sample subjected to proximity ligation in (i), and (iii) sequencing said sequencing library. In some cases, the sequencing reads comprise paired end reads. In some cases, the sequencing reads comprise long reads.

In another aspect, there are provided methods of obtaining a phased human leukocyte antigen (HLA) type of a sample. In some cases, the method comprises aligning a plurality of sequencing reads derived from proximity ligation of nucleic acids in said sample to a reference genome or a variation graph, wherein at least a portion of said plurality of sequencing reads, correspond to a HLA gene locus. In some cases, the method comprises identifying a plurality of single nucleotide polymorphisms (SNPs) and/or indels in said plurality of sequencing reads. In some cases, the method comprises sorting said plurality of SNPs and/or indels into a plurality of phase blocks. In some cases, the method comprises aligning said plurality of sequencing reads to said variation graph to identify a plurality of HLA types. In some cases, the method comprises obtaining a database comprising a plurality of HLA alleles and aligning said plurality of HLA alleles to a reference genome to generate a plurality of SNP signatures of known HLA types. In some cases, the method comprises comparing said plurality of SNPs with said plurality of SNP signatures of known HLA types to identify a plurality of HLA types. In some cases, the method comprises comparing said SNP signature of (e) to said phase blocks of (c) to obtain said phased HLA type. In some cases, the sequencing reads comprise paired end reads. In some cases, the sequencing reads comprise long reads.

In various aspects of methods of obtaining a phased HLA type of a sample provided herein, in some cases said plurality of sequencing reads are obtained by cross-linking said sample, fragmenting nucleic acids in said sample to produce nucleic acid fragments, ligating said nucleic acid fragments to produce ligated nucleic acid fragments, reversing crosslinks, and sequencing said ligated nucleic acid fragments. In some cases, said sample is crosslinked by contacting said sample to a crosslinking agent selected from formaldehyde, psoralen, disuccinimidyl glutarate (DSG), ethylene glycol bis(succinimidyl succinate) (EGS), ultraviolet light, or a combination thereof. In some cases, said fragmenting comprising contacting said sample to an enzyme. In some cases, said enzyme is a nuclease, a restriction endonuclease, a transposase, or a combination thereof. In some cases, said nuclease is a micrococcal nuclease. In some cases, said transposase is Tn5. In some cases, fragmenting comprises non-enzymatic cleavage. In some cases, the method further comprises, subsequent to said ligating, adding a label said nucleic acid fragments. In some cases, said label comprises biotin. In some cases, said label comprises an oligonucleotide. In some cases, said oligonucleotide comprises a barcode. In some cases, said cross-linking links nucleic acids to nucleic acid binding proteins in said sample. In some cases, said plurality of SNPs and/or indels are identified by aligning said sequencing reads to said variation graph. In some cases, the phased HLA type comprises HLA-A, HLA-B, HLA-C, HLA-DRB1, HLA-DRB3, HLA-DRB4, HLA-DRB5, HLA-DQA1, HLA-DQB1, HLA-DPA1, and/or HLA-DPB1. In some cases, the sample comprises nucleic acids enriched for HLA sequences. In some cases, the entire HLA region is phased with over 90% of the phased SNPs in a single phase block. In some cases, major and minor HLA type groups are phased. In some cases, the method is completed in less than 30 hours of CPU time. In some cases, the method is completed in less than 15 hours, less than 10 hours, less than 5 hours, less than 4 hours, less than 3 hours, less than 2 hours, or less than 1 hour of CPU time. In some cases, the method is completed in about 30 to about 40 minutes of CPU time. In some cases, the sequencing reads comprise paired end reads. In some cases, the sequencing reads comprise long reads.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

An understanding of the features and advantages of the present invention will be obtained by reference to the following detailed description that sets forth illustrative embodiments, in which the principles of the invention are utilized, and the accompanying drawings of which:

FIG. 1 shows a flow diagram for one method of phased HLA typing.

FIG. 2 shows a flow diagram for one method of obtaining sequencing reads.

FIG. 3 illustrates read pair linkage for separation of maternal and paternal read pairs.

FIG. 4 shows an example phased HLA typing.

FIG. 5 shows a flow diagram for one method of phased HLA typing.

FIG. 6 illustrates utilization of capture probes in phased HLA haplotypes.

FIG. 7 phasing HLA region using proximity ligation.

FIG. 8 shows a computer system that is programmed or otherwise configured to implement methods provided herein.

### DETAILED DESCRIPTION

HLA matching is an important factor for matching donors and recipients for transplantation. For unrelated donors and recipients, matching is generally done by typing at certain HLA loci. However, matching at the haplotype level may provide better clinical outcomes.

HLA genes have high degrees of polymorphism and complex patterns of association. This creates a challenge for determining HLA haplotypes over multiple loci.

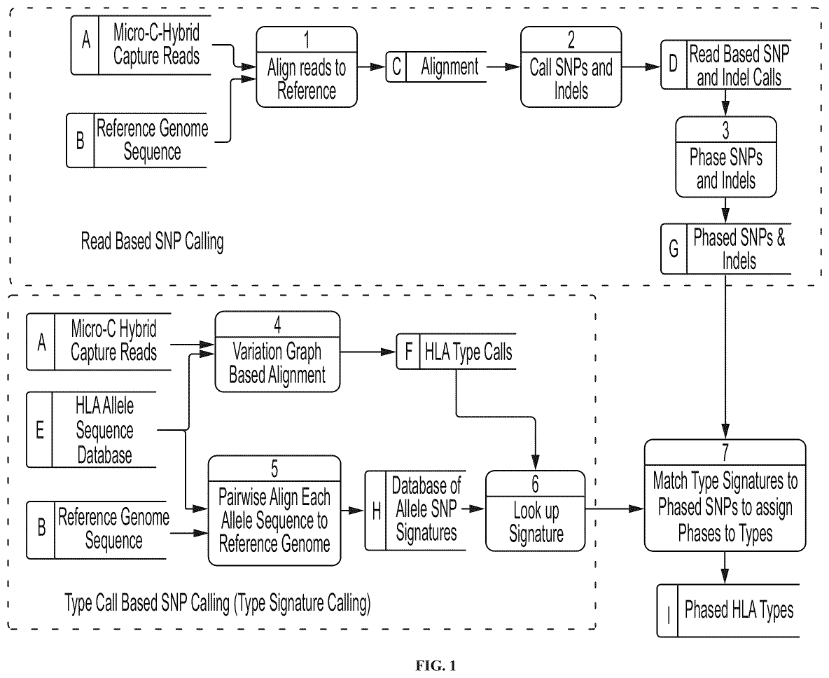

This disclosure provides an approach for producing phased HLA types. Long-range haplotype phasing techniques can be combined with HLA genotype calling to produce phased HLA regions. For example, as shown in FIG. 1, phased HLA typing can combine two independent pipelines to produce the final phased output. The first pipeline (FIG. 1, upper panel) calls and phases single nucleotide polymorphisms (SNPs) from high throughput proximity ligation reads. The second pipeline (FIG. 1, lower panel) calls HLA types from the same data but also using a database of known HLA alleles, then aligns the closest matching sequence from each called HLA allele type to the reference genome to generate a SNP signature for that allele. The SNP signature from the second process (FIG. 1, lower panel) is then matched to the high-quality phased SNPs from the first process (FIG. 1, upper panel) to determine if the SNP signature more closely matches parent 1 SNPs or parent 2 SNPs. Each allele is then assigned to the parental chromosome of the closest matching phased SNP set.

Provided herein are methods of obtaining a phased human leukocyte antigen (HLA) type of a sample. Such methods can comprise aligning a plurality of sequencing reads to a reference genome, wherein at least a portion of said plurality of sequencing reads correspond to an HLA gene locus. Next, the method can comprise identifying a plurality of single nucleotide polymorphisms (SNPs) and/or indels in said plurality of sequencing reads. Alternatively, or in combination, the method can comprise identifying said plurality of SNPs by comparing said plurality of sequencing reads to a variation graph. Then the method can comprise sorting said plurality of SNPs and indels into phase blocks. The method can then comprise aligning said plurality of sequencing reads to a variation graph to identify a plurality of HLA types. Next the method can comprise comparing said plurality of HLA types to a plurality of known HLA alleles to obtain a SNP signature for each HLA allele. Then the method can comprise comparing said SNP signature of (e) to said phase blocks of (c) to obtain said phased HLA type.

Sequencing reads can be paired end reads, single short reads, long reads (e.g., via nanopore, SMRT, HiFi) or any other suitable sequence read format.

In some cases, sequencing reads are obtained using any suitable proximity ligation method such as Hi-C, Chicago, Micro-C, or Omni-C. In some cases, sequencing reads are obtained using Micro-C. The proximity ligation reads can be paired end reads, with each read of the pair providing sequence information of one end or the other of both sides of a proximity ligation site. The proximity ligation reads can be long reads, with one long read providing sequence information of both sides of a proximity ligation site, or even sequence information surrounding multiple proximity ligations sites in one concatemer. The sequencing reads can be aligned to a reference genome using a read aligner (e.g., BWA aligner). Alternatively, or in combination, the sequencing reads can be aligned to a variation graph, such as a variation graph constructed by Kourami. SNPs and indels are called based on the alignment using any suitable software, for example DeepVariant or Kourami. In some cases, it is desired to use alignment to the variation graph in cases where alignment to a fixed reference can result in misaligned reads in homologous regions. In particular cases, HLA-B and HLA-C have regions of high similarity that can sometimes result in reads from HLA-C mistakenly aligned to HLA-B, causing true SNPs and indels not to be called.

Once SNPs and indels are obtained, they can be sorted into phase consistent blocks using maximum-likelihood based software. In some cases, the maximum-likelihood based software is Hapcut2. The maximum-likelihood based software assigns the SNPs and indels to phase blocks. Each phase block can contain a set of SNPs and indels labeled P1 or P2 for whether they derive from parental chromosome P1 or parental chromosome P2. When using paired end reads, read sizes can vary from a few hundred bases to chromosome scale, therefore, phase blocks are often obtained that span the entire 4 Mbp HLA region with over 90% of the phased SNPs in the single largest phase block.

In some cases, HLA allele types are called using hybrid capture proximity ligation reads. The reads are aligned to a variation graph constructed from the IPD-IMGT/HLA database of HLA variants. By examining these alignments, the ideal pair of HLA types are identified in the variation graph. Each HLA type is a tag of the form, such as, for example, A*26:01:02:01 describing the major and minor HLA type groups for the allele. HLA*LA or Kourami can be used for this purpose or alternatively, these steps can be assembled independently.

Next, in some cases, a HLA type signature database can be built by aligning each sequence in the database of sequences of known HLA alleles to the corresponding region of a reference genome assembly and analyzing the alignment to produce a list of SNPs and indels for each allele, thereby creating a SNP signature for each allele. In some cases, the allele type calls from alignment to the variation graph can be used to determine the SNP signatures for the sample being analyzed to produce a list of SNP signatures for the sample. Next, each SNP in the SNP signature can be compared, both sequence and location, to the phased SNPs in that region. The phase, P1 or P2, with the most matching SNPs and indels can be assigned to that allele. The result of this analysis conducted on all of the alleles is a phased set of HLA types. In some cases HLA types are called directly by matching the allele type signature database to the phased SNPs and indels from the maximum-likelihood analysis.

In some cases, local phased assembly for each gene is performed using the capture reads. In this case, the local phased assembly of each individual gene can be used in the same manner as the called type sequences (e.g., matching SNPs in the local phased assembly sequence to SNPs called with Deep Variant and phased with Hapcut2). In some cases, this approach is advantageous when the individual sample is significantly diverged from even the closest match in the database of known HLA alleles. In addition, there can be a performance advantage since the graph alignment step requires only 30 wall clock hours of CPU time to compute while local phased assemblies of each gene would be expected to require less time. In some cases, the graph alignment step requires only 30-40 wall clock minutes of CPU time to compute.

### Long Range Haplotype Phasing

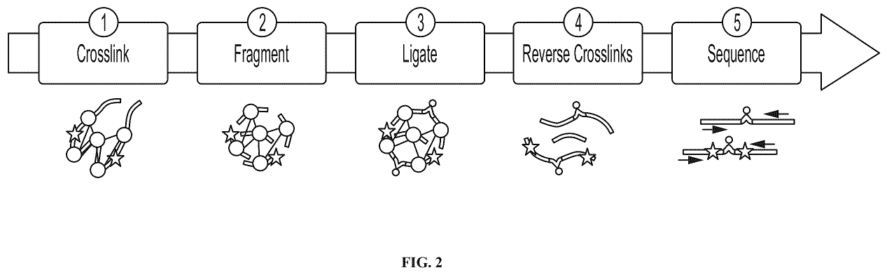

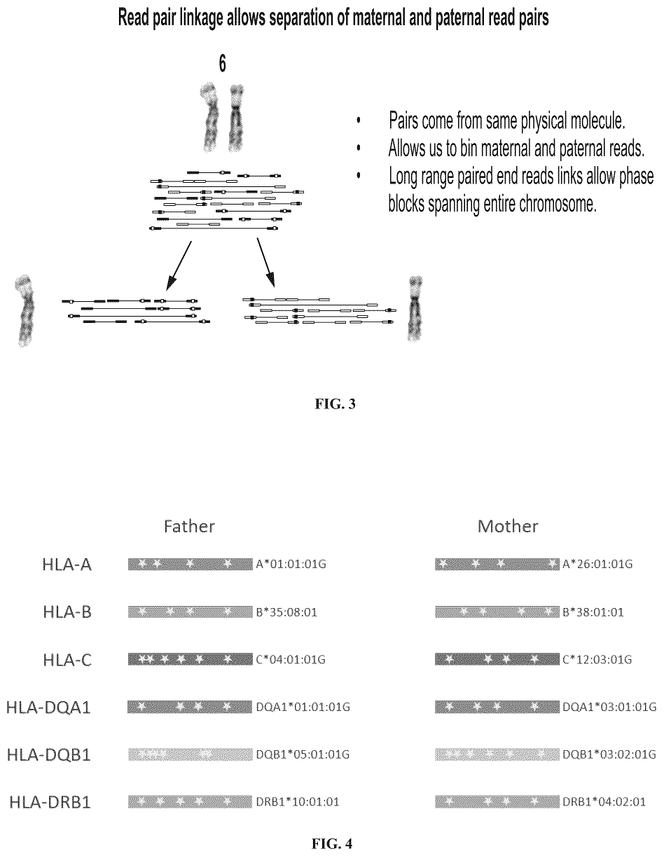

Disclosed herein are methods for generating read sets, including phased read-sets, for applications including genome assembly and haplotype phasing, using long-read or short-read sequencing technologies. Exemplary techniques include but are not limited to proximity ligation techniques such as Hi-C, Chicago, Micro-C, and Omni-C. Nucleic acid molecules can be bound (e.g., in a chromatin structure), cleaved to expose internal ends, re-attached at junctions to other exposed ends, freed from binding, and sequenced. This technique can produce nucleic acid molecules comprising multiple sequence segments. The multiple sequence segments within a nucleic acid molecule can have phase information preserved while being rearranged relative to their natural or starting position and orientation. Sequence segments on either side of a junction can be confidently considered to come from the same phase of a sample nucleic acid molecule. In an example, FIG. 2 shows the steps of crosslinking a chromatin structure, fragmenting the nucleic acids in the chromatin, ligating, or otherwise connecting exposed ends, reversing the crosslinking, and sequencing the resulting nucleic acids. As shown in FIG. 3, linkage of read information from two or more connected regions of a nucleic acid generated in this way can indicate that read information came from the same original nucleic acid molecule, allowing binning of maternal and paternal reads, and enabling phased blocks of sequence even spanning an entire chromosome.

Nucleic acid molecules, including high molecular weight DNA, can be bound or immobilized on at least one nucleic acid binding moiety. For example, DNA assembled into in vitro chromatin aggregates and fixed with formaldehyde treatment are consistent with methods herein. Nucleic acid binding or immobilizing approaches include, but are not limited to, in vitro or reconstituted chromatin assembly, native chromatin, DNA-binding protein aggregates, nanoparticles, DNA-binding beads, or beads coated using a DNA-binding substance, polymers, synthetic DNA-binding molecules or other solid or substantially solid affinity molecules. In some cases, the beads are solid phase reversible immobilization (SPRI) beads (e.g., beads with negatively charged carboxyl groups such as Beckman-Coulter Agencourt AMPure XP beads).

Nucleic acids bound to a nucleic acid binding moiety such as those described herein can be held such that a nucleic acid molecule having a first segment and a second segment separated on the nucleic acid molecule by a distance greater than a read distance on a sequencing device (10 kb, 50 kb, 100 kb or greater, for example) are bound together independent of their common phosphodiester bonds. Upon cleavage of such a bound nucleic acid molecule, exposed ends of the first segment and the second segment may ligate to one another. In some cases, the nucleic acid molecules are bound at a concentration such that there is little or no overlap between bound nucleic acid molecules on a solid surface, such that exposed internal ends of cleaved molecules are likely to re-ligate or become reattached only to exposed ends from other segments that were in phase on a common nucleic acid source prior to cleavage. Consequently, a DNA molecule can be cleaved and cleaved exposed internal ends can be re-ligated, for example at random, without loss of phase information.

A bound nucleic acid molecule can be cleaved to expose internal ends through one of any number of enzymatic and non-enzymatic approaches. For example, a nucleic acid molecule can be digested using a restriction enzyme, such as a restriction endonuclease that leaves a single stranded overhang. MboI digest, for example, is suitable for this purpose, although other restriction endonucleases are contemplated. Lists of restriction endonucleases are available, for example, in most molecular biology product catalogues. Other non-limiting techniques for nucleic acid cleavage include using a transposase, tagmentation enzyme complex, topoisomerase, nonspecific endonuclease, DNA repair enzyme, RNA-guided nuclease, fragmentase, or alternate enzyme. Transposase, for example, can be used in combination with unlinked left and right borders to create a sequence-independent break in a nucleic acid that is marked by attachment of transposase-delivered oligonucleotide sequence. Physical means can also be used to generate cleavage, including mechanical means (e.g., sonication, shear), thermal means (e.g., temperature change), or electromagnetic means (e.g., irradiation, such as UV irradiation).

Immobilization of nucleic acids at this stage can keep the cleaved nucleic acid molecule fragments in close physical proximity, such that phase information for the initial molecule is preserved. A benefit of the fixation, e.g. to chromatin aggregates, is that separate regions of a common nucleic acid molecule can be held together independent of their phosphodiester backbone, such that their phase information is not lost upon cleavage of the phosphodiester backbone. This benefit is also conveyed through alternate scaffolds to which a nucleic acid molecule is attached prior to cleavage.

Optionally, single stranded “sticky” end overhangs are modified to prevent reannealing and re-ligation. For example, sticky ends are partially filled-in, such as by adding one nucleotide and a polymerase. In this way, the entire single-stranded end cannot be filled in, but the end is modified to prevent re-ligation with a formerly complementary end. In the example of MboI digestion, which leaves a 5′ GATC 5-prime overhang, only the Guanosine nucleotide triphosphate is added. This results in only a “G” fill-in of the first complementary base (“C”) and result in a 5′ GAT overhang. This step renders the free sticky ends incompatible for re-ligation to one another, but preserves sticky ends for downstream applications. Alternately, blunt ends are generated through completely filling in the overhangs, restriction digest with blunt-end generating enzymes, treatment with a single-strand DNA exonuclease, or nonspecific cleavage. In some cases, a transposase is used to attach adapter ends having blunt or sticky ends to the exposed internal ends of the DNA molecule.

Optionally, a “punctuation oligonucleotide” is introduced. This punctuation oligonucleotide marks cleavage/re-ligation sites. Some punctuation oligonucleotides have single-stranded overhangs on both ends that are compatible with the partially filled-in overhangs generated on the exposed nucleic acid sample internal ends. An example of a punctuation oligonucleotide is shown below. In some cases, the double-stranded oligonucleotide having single-stranded overhangs is modified, such as by 5′ phosphate removal at its 5′ ends, so that it cannot form concatemers during ligation. Alternately, blunt punctuation oligonucleotides are used, or cleavage sites are not marked using a distinct punctuation oligonucleotide.

In some systems, such as when a transposase is used, punctuation is accomplished through addition of transpososome border sequences, followed by ligation of border sequences to one another or to a punctuation oligo. An exemplary punctuation oligo is presented below. However, alternate punctuation oligos are consistent with the disclosure herein, varying in sequence, length, overhang presence or sequence, or modification such as 5′ de-phosphorylation.

In some cases, the double-stranded region of the punctuation oligonucleotide will vary. A relevant feature of the punctuation oligonucleotide is the sequence of its overhang, allowing ligation to the nucleic acid sample but optionally modified precluding auto-ligation or concatemer formation. It is often preferred that the punctuation oligonucleotide comprise sequence that does not occur or is less likely to occur in a target nucleic acid molecule, such that it is easily identified in a downstream sequence reaction. Punctuation oligos are optionally barcoded, for example with a known barcode sequence or with a randomly generated unique identifier sequence. Unique identifier sequences can be designed to make it highly unlikely for multiple junctions in a nucleic acid molecule or in a sample to be barcoded with the same unique identifier.

Cleaved ends can be attached to one another directly or through an oligo (e.g., a punctuation oligo), for example using a ligase or similar enzyme. Ligation can proceed such that the free single-stranded ends of an immobilized high-molecular weight nucleic acid molecule are ligated directly or to the punctuation oligonucleotide. Because the punctuation oligonucleotide, if utilized, can have two ligatable ends, this ligation can effectively chain regions of the high molecular weight nucleic acid molecule together. Alternative approaches resulting in affixing a punctuating sequence or molecule between two exposed ends can also be employed, as can approaches for directly connecting two exposed ends without punctuation.

Nucleic acids can then be liberated from the nucleic acid binding moiety. In the case of in vitro chromatin aggregates, this can be accomplished by reversing the cross-links, or digesting the protein components, or both reversing the crosslinking and digesting protein components. A suitable approach is treatment of complexes with proteinase K, though many alternatives are also contemplated. For other binding techniques, suitable methods can be employed, such as the severing of linker molecules or the degradation of a substrate.

Nucleic acid molecules resulting from such techniques can have a variety of relevant features. Sequence segments within a nucleic acid molecule can be rearranged relative to their natural or starting positions and orientations, but with phase information preserved. Consequently, sequence segments on either side of a junction can be confidently assigned to a common phase of a common sample molecule. Thus, segments far removed from one another on a molecule can be, by such techniques, brought together or in proximity such that portions or the entirety of each segment is sequenced in a single run of a single molecule sequencing device, allowing definitive phase assignment. Alternately, in some cases originally adjacent segments can become separated from one in the resultant nucleic acid. In some cases, the nucleic acid molecules can be re-ligated such that at least about 50%, 55%, 60%, 65%, 70%, 75%, 80%, 85%, 90%, 91%, 92%, 93%, 94%, 95%, 96%, 97%, 98%, 99%, 99.9%, 99.99%, 99.999%, or 100% of re-ligations are between segments that were in phase on a common nucleic acid source prior to cleavage.

Another relevant feature of the resultant molecules is that, in some cases, most or all the original molecular sequence is preserved, though perhaps rearranged, in the final punctuated or rearranged molecule. For example, in some cases no more than 1%, 2%, 3%, 4%, 5%, 10%, 15%, or 20% of the original molecule is lost in producing the resultant molecule or molecules. Consequently, in addition to being useful as a phase determinant, the resultant molecule retains a substantial proportion of the original molecule sequence, such that the resultant molecule is optionally used to concurrently generate sequence information such as contig information useful in de novo sequencing or as independent verification of previously generated contig information.

Another feature of libraries of some resultant molecules is that cleavage junctions are not common to multiple members of a population of resultant molecules. That is, that different copies of the same starting nucleic acid molecule can end up with different patterns of junction and rearrangement. Random cleavage junctions can be generated with a non-specific cleavage molecule, or through variation in restriction endonuclease selection or digestion parameters.

A consequence of having molecule-specific cleavage sites is that in some cases punctuation oligonucleotides are optionally excluded from the process that results in the ‘punctuation molecule’ re-shuffling and re-ligation to no ill effect. By aligning segments of three or more reshuffled molecules, one observes that cleavage sites are readily identified by their absence in the majority of other members of a library. That is, when three or more reshuffled molecules are locally aligned, a segment can be found to be common to all of the molecules, but the edges of the segment can vary among the molecules. By noting where segment local sequence similarity ends, one can map cleavage junctions in an ‘unpunctuated’ rearranged nucleic acid molecule.

The resulting nucleic acid molecules can be sequenced, for example on a long-read sequencer. The resulting sequence reads contain segments that alternate between nucleic acid sequence from the original input molecule and, if they are used, sequences of the punctuation oligo. These reads can be processed by a computer to split sequence data from each read using the punctuation oligonucleotide sequence, or are otherwise processed to identify junctions. The sequence segments within each read can be segments from a single input high molecular weight DNA molecule. The original nucleic acid molecule can comprise a genome sequence or fraction thereof, such as a chromosome. The sets of segment reads can be discontinuous in the original nucleic acid molecule but reveal long-range, haplotype-phased data. These data can be used for de novo genome assembly and phasing heterozygous positions in the input genome. Sequence between junctions indicates contiguous nucleic acid sequence in the source nucleic acid sample, while sequence across a junction is indicative of a nucleic acid segment that is in phase in the nucleic acid sample but that may be far removed in the arranged scaffold from the adjacent segment.

Junctions can be identified by a variety of approaches. If punctuation oligos are used, junctions can be identified at reads containing the punctuation oligo sequence. Alternately, junctions can be identified by comparison to a second sequence source (and, preferably, a third sequence source) for a nucleic acid molecule, such as a previously generated contig sequence dataset or a second, independently generated DNA chain molecule having independently derived junctions. As the sequence is aligned, for example, the quality or confidence of alignment to a particular location can indicate where one segment ends and another begins. If restriction enzymes are used to generate cleavages, sequences containing the restriction enzyme recognition site can be evaluated for potentially containing a junction. Note that not every restriction enzyme recognition site may contain a junction, as some restriction enzyme recognition sites may not have been physically accessible by the enzyme while the nucleic acid was bound to the support, for example. Statistical information can also be employed in identifying junctions; for example, the length segments between junctions may be predicted to be of a certain average value or to follow a certain distribution.

A benefit of the manipulations herein is that they can preserve molecular phase information while bringing nonadjacent regions of the molecule in proximity such that they are included in a single nucleic acid molecule at a distance suitable for sequencing in a single read, such as a long read. Thus, regions that are separated in the starting sample by greater than the distance of a single long read operation (for example 10 kb, 15 kb, 20 kb, 30 kb, 50 kb, 100 kb or greater) are brought into local proximity such that they are within the distance covered by a single read of a long-range sequencing reaction. Thus, regions that are separated by more than the range of the sequencing technology for a single read in the original sample are read in a single reaction in the phase-preserved, rearranged molecule.

Resultant rearranged molecules can be sequenced and their sequence information mapped to independently or concurrently generated sequence reads or contig information, or to a known reference genome sequence (for example, the known sequence of the human genome). Segments adjacent on the resultant rearranged molecule reads are presumed to be in phase. Accordingly, when these segments are mapped to disparate contigs or long range sequence reads, the reads are assigned to a common phase of a common molecule in the sequence assembly.

Alternately, if multiple independently generated resultant rearranged molecules are sequenced concurrently, phased sample data is optionally generated from these molecules alone, such that segment sequences separated by junctions are inferred to be in phase, while sequences not separated by junctions are inferred to represent stretches of nucleic acids contiguous in the sample itself and useful for, for example, de novo sequence determination as well as being useful for phase determination. However, additionally or as an alternative, multiple independently generated resultant rearranged molecules sequenced concurrently can still be compared to independently generated scaffold or contig information

Methods and compositions presented herein can preserve long-range phase information, particularly for molecule segments separated by greater than the length of a read in a sequencing technology (10 kb, 20 kb, 50 kb, 100 kb, 500 kb or greater, for example), while providing such nonadjacent segments in a rearranged or often ‘punctuated’ molecule where the segments are adjacent or close enough to be covered by a single read.

In some instances, resultant rearranged molecules are combined with native molecules for sequencing. The native molecules can be recognized and utilized informatically by the lack of punctuation sequences, if employed. Native molecules are sequenced using short or long read technology, and their assembly is guided by the phase information and segment sequence information generated through sequencing of the rearranged molecule or library.

### Hanlotype Phasing

In diploid genomes, it often important to know which allelic variants are linked on the same chromosome. This is known as the haplotype phasing. Short reads from high-throughput sequence data rarely allow one to directly observe which allelic variants are linked. Computational inference of haplotype phasing can be unreliable at long distances. The disclosure provides one or more methods that allow for determining which allelic variants are linked using allelic variants on read pairs. In some cases, phasing with methods of the present disclosure is conducted without imputation.

In various embodiments, the methods and compositions of the disclosure enable the haplotype phasing of diploid or polyploid genomes with regard to a plurality of allelic variants. The methods described herein can thus provide for the determination of linked allelic variants that are linked based on variant information from read pairs and/or assembled contigs using the same. Examples of allelic variants include, but are not limited to, those that are known from the 1000 genomes, UK10K, HapMap and other projects for discovering genetic variation among humans. Disease association to a specific gene can be revealed more easily by having haplotype phasing data as demonstrated, for example, by the finding of unlinked, inactivating mutations in both copies of SH3TC2 leading to Charcot-Marie-Tooth neuropathy (Lupski J R, Reid J G, Gonzaga-Jauregui C, et al. N. Engl. J. Med. 362:1181-91, 2010) and unlinked, inactivating mutations in both copies of ABCG5 leading to hypercholesterolemia 9 (Rios J, Stein E, Shendure J, et al. Hum. Mol. Genet. 19:4313-18, 2010).

Humans are heterozygous at an average of 1 site in 1,000. In some cases, a single lane of data using high-throughput sequencing methods can generate at least about 150,000,000 read pairs. Read pairs can be about 100 base pairs long. From these parameters, one-tenth of all reads from a human sample is estimated to cover a heterozygous site. Thus, on average one-hundredth of all read pairs from a human sample is estimated to cover a pair of heterozygous sites. Accordingly, about 1,500,000 read pairs (one-hundredth of 150,000,000) provide phasing data using a single lane. With approximately 3 billion bases in the human genome, and one in one-thousand being heterozygous, there are approximately 3 million heterozygous sites in an average human genome. With about 1,500,000 read pairs that represent a pair of heterozygous sites, the average coverage of each heterozygous site to be phased using a single lane of a high-throughput sequence method is about (1×), using a typical high-throughput sequencing machine. A diploid human genome can therefore be reliably and completely phased with one lane of a high-throughput sequence data relating sequence variants from a sample that is prepared using the methods disclosed herein. In some examples, a lane of data can be a set of DNA sequence read data. In further examples, a lane of data can be a set of DNA sequence read data from a single run of a high-throughput sequencing instrument.

As the human genome consists of two homologous sets of chromosomes, understanding the true genetic makeup of an individual requires delineation of the maternal and paternal copies or haplotypes of the genetic material. Obtaining a haplotype in an individual is useful in several ways. First, haplotypes are useful clinically in predicting outcomes for donor-host matching in organ transplantation and are increasingly used as a means to detect disease associations. Second, in genes that show compound heterozygosity, haplotypes provide information as to whether two deleterious variants are located on the same allele, greatly affecting the prediction of whether inheritance of these variants is harmful. Third, haplotypes from groups of individuals have provided information on population structure and the evolutionary history of the human race. Lastly, recently described widespread allelic imbalances in gene expression suggest that genetic or epigenetic differences between alleles may contribute to quantitative differences in expression. An understanding of haplotype structure will delineate the mechanisms of variants that contribute to allelic imbalances.

In certain embodiments, the methods disclosed herein comprise an in vitro technique to fix and capture associations among distant regions of a genome as needed for long-range linkage and phasing. In some cases, the method comprises constructing and sequencing an XLRP library to deliver very genomically distant read pairs. In some cases, the interactions primarily arise from the random associations within a single DNA fragment. In some examples, the genomic distance between segments can be inferred because segments that are near to each other in a DNA molecule interact more often and with higher probability, while interactions between distant portions of the molecule will be less frequent. Consequently, there is a systematic relationship between the number of pairs connecting two loci and their proximity on the input DNA. The disclosure can produce read pairs capable of spanning the largest DNA fragments in an extraction. The input DNA for this library had a maximum length of 150 kbp, which is the longest meaningful read pair observed from the sequencing data. This suggests that the present method can link still more genomically distant loci if provided larger input DNA fragments. By applying improved assembly software tools that are specifically adapted to handle the type of data produced by the present method, a complete genomic assembly may be possible.

Extremely high phasing accuracy can be achieved by the data produced using the methods and compositions of the disclosure. In comparison to previous methods, the methods described herein can phase a higher proportion of the variants. Phasing can be achieved while maintaining high levels of accuracy. The techniques herein can allow for phasing at an accuracy of greater than about 70%, 80%, 90%, 95%, 96%, 97%, 98%, 99%, 99.9%, 99.99%, or 99.999%. The techniques herein can allow for accurate phasing with less than about 500× sequencing depth, 450× sequencing depth, 400× sequencing depth, 350× sequencing depth, 300× sequencing depth, 250× sequencing depth, 200× sequencing depth, 150× sequencing depth, 100× sequencing depth, or 50× sequencing depth. This phase information can be extended to longer ranges, for example, greater than about 200 kbp, about 300 kbp, about 400 kbp, about 500 kbp, about 600 kbp, about 700 kbp, about 800 kbp, about 900 kbp, about 1 Mbp, about 2 Mbp, about 3 Mbp, about 4 Mbp, about 5 Mbp, or about 10 Mbp. In some embodiments, more than 90% of the heterozygous SNPs for a human sample can be phased at an accuracy greater than 99% using less than about 250 million reads or read pairs, e.g., by using only 1 lane of Illumina HiSeq data. In other cases, more than about 40%, 50%, 60%, 70%, 80%, 90%, 95%, or 99% of the heterozygous SNPs for a human sample can be phased at an accuracy greater than about 70%, 80%, 90%, 95%, 96%, 97%, 98%, 99%, 99.9%, 99.99%, or 99.999% using less than about 250 million or about 500 million reads or read pairs, e.g., by using only 1 or 2 lanes of Illumina HiSeq data. For example, more than 95% or 99% of the heterozygous SNPs for a human sample can be phase at an accuracy greater than about 95% or 99% using less about 250 million or about 500 million reads. In further cases, additional variants can be captured by increasing the read length to about 200 bp, 250 bp, 300 bp, 350 bp, 400 bp, 450 bp, 500 bp, 600 bp, 800 bp, 1000 bp, 1500 bp, 2 kbp, 3 kbp, 4 kbp, 5 kbp, 10 kbp, 20 kbp, 50 kbp, or 100 kbp.

In other embodiments of the disclosure, the data from an XLRP library can be used to confirm the phasing capabilities of the long-range read pairs. The accuracy of those results is on par with the best technologies previously available, but further extending to significantly longer distances. The current sample preparation protocol for a particular sequencing method recognizes variants located within a read-length, e.g., 150 bp, of a targeted site for phasing. In one example, from an XLRP library built for NA12878, a benchmark sample for assembly, 44% of the 1,703,909 heterozygous SNPs present were phased with an accuracy greater than 99%. In some cases, this proportion can be expanded to nearly all variable sites with the judicious choice of enzymes or with digestion conditions.

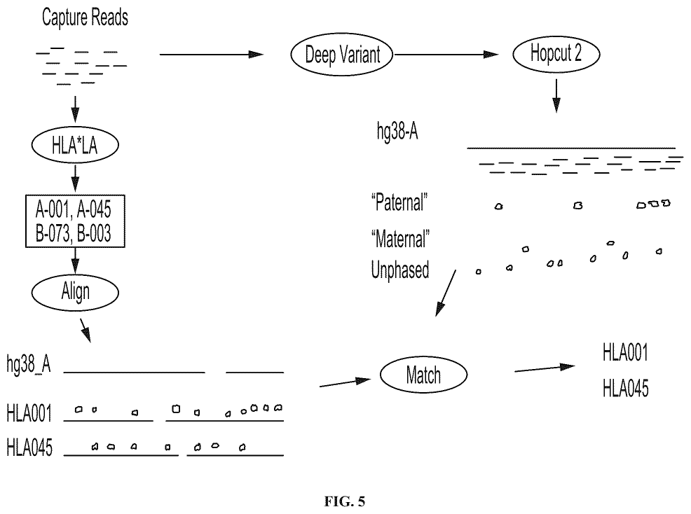

Haplotype phasing can include phasing the human leukocyte antigen (HLA) region (e.g., Class I HLA-A, B, and C; Class II HLA-DRB1/3/4/5, HLA-DQA1, HLA-DQB1, HLA-DPA1, HLA-DPB1). FIG. 4 shows exemplary phased HLA genotypes. The HLA region of the genome is densely polymorphic and can be difficult to sequence or phase with standard sequencing approaches. Techniques of the present disclosure can provide for improved sequencing and phasing accuracy of the HLA region of the genome. Using techniques of the present disclosure, the HLA region of the genome can be phased accurately as part of phasing larger regions (e.g., chromosome arms, chromosomes, whole genomes) or on its own (e.g., by targeted enrichment such as hybrid capture). In an example, the HLA region on its own was phased accurately at a sequencing depth of approximately 300×. These techniques can provide advantages over traditional approaches for HLA analysis, such as long-range PCR; for example, long-range PCR can involve complex protocols and many separate reactions. As discussed further herein, samples can be multiplexed for sequencing analysis, for example by including sample-identifying barcodes in bridge oligonucleotides or elsewhere, and de-multiplexing the sequence information based on the barcodes. In an example, multiple samples are subjected to proximity ligation, barcoded with sample-identifying barcodes (e.g., in the bridge oligonucleotide), the HLA region is targeted (e.g., by hybrid capture), and multiplexed sequencing is conducted, allowing phasing of the HLA region for multiple samples. In some cases, phasing the HLA region is conducted without imputation.

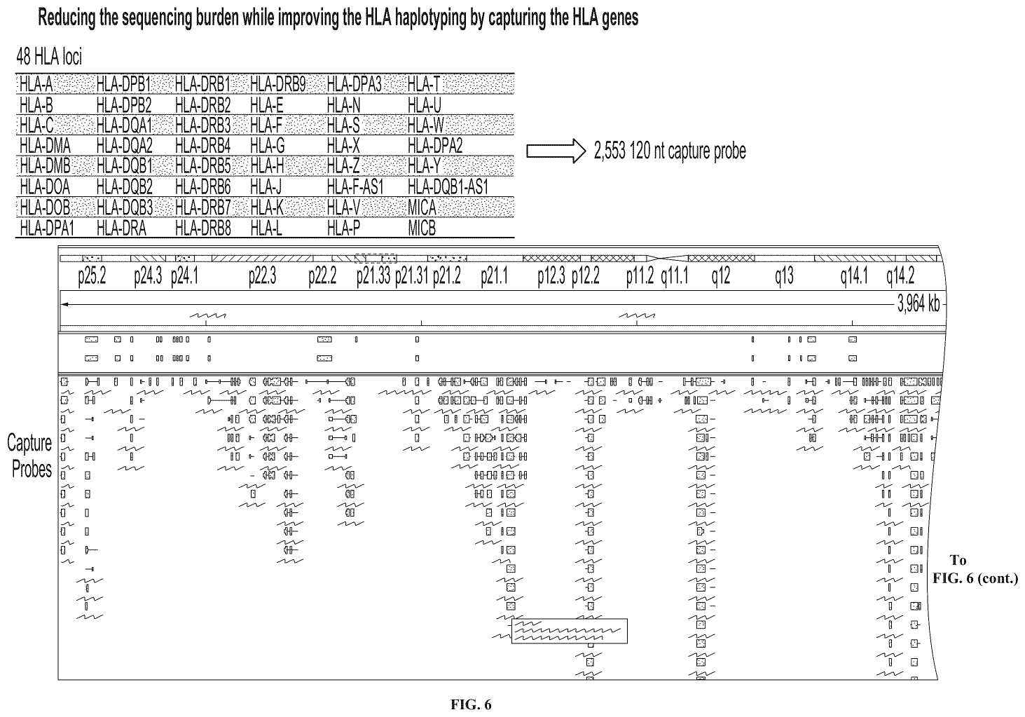

In an example, as shown in FIG. 5, sequencing reads from a library prepared as disclosed herein can be called for variants (e.g., using Deep Variant or other approaches) and phased (e.g., using Hapcut2 or other approaches). This can produce phased sets of variants, which can be referred to as “paternal” and “maternal” (as well as any remaining unphased variants). The same sequencing reads can also be HLA typed (e.g., using HLA*LA (Dilthey, et al. HLA*LA-HLA typing from linearly projected graph alignments. Bioinformatics 35(21) 2019, 4394-4396, which is hereby incorporated by reference in its entirety), Kourami (Lee, et al. Kourami: graph-guided assembly for novel human leukocyte antigen allele discovery. Genome Biology 19(1) 2018, 16, which is hereby incorporated by reference in its entirety), or other approaches) and aligned. The phased variants and the aligned HLA types can then be matched together to provide phased HLA types.

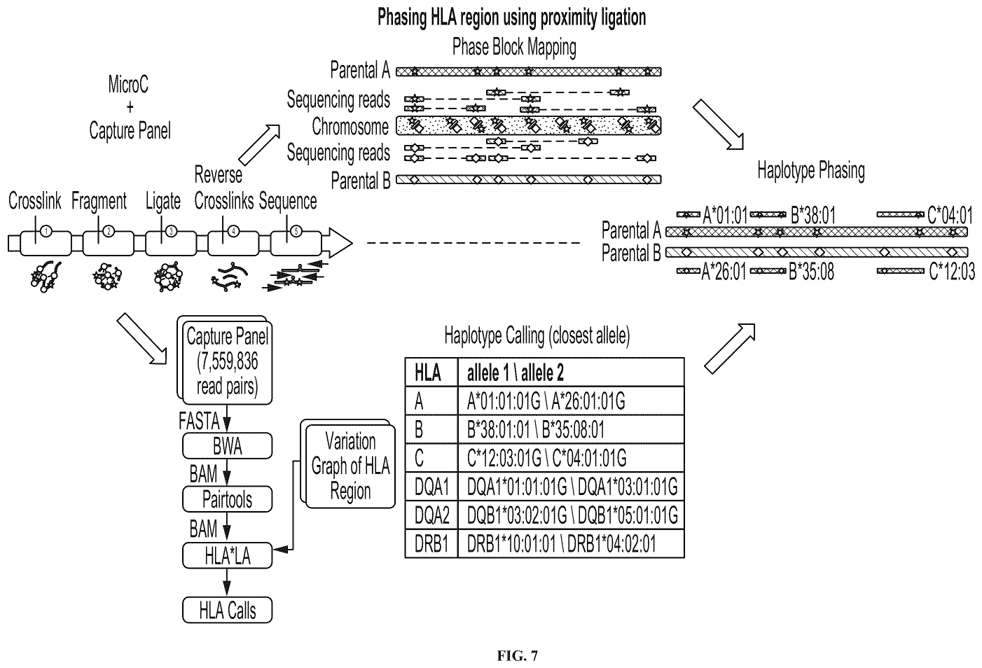

In another example, as shown in FIG. 7, a proximity ligation approach is used to produce a phased HLA typed analysis. Micro-C is used to prepare a proximity ligation library which is made target-specific for the HLA region using a capture panel (as further discussed herein). Sequencing reads from the library are used to perform phase block mapping and also to perform haplotype calling (e.g., to the closest allele). Phase block maps and haplotype calls are then combined to produce a phased haplotype of the HLA region. In addition to calling haplotypes to the closest allele, haplotypes that do not match any previously known allele can also be noted (instead of or in addition to calling the known allele closest to such a haplotype).

### Target Enrichment

In certain embodiments, the disclosure provides methods for the enrichment of HLA nucleic acids for determining a phased HLA haplotype. In some cases, the methods for enrichment are in a solution-based format. In some cases, the target nucleic acid can be labeled with a labeling agent. In other cases, the target nucleic acid can be crosslinked to one or more association molecules that are labeled with a labeling agent. Examples of labeling agents include, but are not limited to, biotin, polyhistidine tags, and chemical tags (e.g., alkyne and azide derivatives used in Click Chemistry methods). Further, the labeled target nucleic acid can be captured and thereby enriched by using a capturing agent. The capturing agent can be streptavidin and/or avidin, an antibody, a chemical moiety (e.g., alkyne, azide), and any biological, chemical, physical, or enzymatic agents used for affinity purification.

In some cases, immobilized or non-immobilized nucleic acid probes can be used to capture the target nucleic acids. For example, the target nucleic acids can be enriched from a sample by hybridization to the probes on a solid support or in solution. In some examples, the sample can be a genomic sample. In some examples, the probes can be an amplicon. The amplicon can comprise a predetermined sequence. Further, the hybridized target nucleic acids can be washed and/or eluted from the probes. The target nucleic acid can be a DNA, RNA, cDNA, or mRNA molecule.

In some cases, the enrichment method can comprise contacting the sample comprising the target nucleic acid to the probes and binding the target nucleic acid to a solid support. In some cases, the sample can be fragmented using enzymatic methods to yield the target nucleic acids. In some cases, the probes can be specifically hybridized to the target nucleic acids. In some cases, the target nucleic acids can have an average size of about 145 bp to about 600 bp, about 100 bp to about 2500 bp, about 600 to about 2500 bp, or about 350 bp to about 1000 bp. The target nucleic acids can be further separated from the unbound nucleic acids in the sample. The solid support can be washed and/or eluted to provide the enriched target nucleic acids. In some examples, the enrichment steps can be repeated for about 1, 2, 3, 4, 5, 6, 7, 8, 9, or 10 times. For example, the enrichment steps can be repeated for about 1, 2, or 3 times.

In some cases, the enrichment method can comprise providing probe derived amplicons wherein said probes for amplification are attached to a solid support. The solid support can comprise support-immobilized nucleic acid probes to capture specific target nucleic acid from a sample. The probe derived amplicons can hybridize to the target nucleic acids. Following hybridization to the probe amplicons, the target nucleic acids in the sample can be enriched by capturing (e.g., via capturing agents as biotin, antibodies, etc.) and washing and/or eluting the hybridized target nucleic acids from the captured probes. The target nucleic acid sequence(s) may be further amplified using, for example, PCR methods to produce an amplified pool of enriched PCR products.

In some cases, the solid support can be a microarray, a slide, a chip, a microwell, a column, a tube, a particle, or a bead. In some examples, the solid support can be coated with streptavidin and/or avidin. In other examples, the solid support can be coated with an antibody. Further, the solid support can comprise a glass, metal, ceramic or polymeric material. In some embodiments, the solid support can be a nucleic acid microarray (e.g., a DNA microarray). In other embodiments, the solid support can be a paramagnetic bead.

In particular embodiments, the disclosure provides methods for amplifying the enriched DNA. In some cases, the enriched DNA is a read-pair. The read-pair can be obtained by the methods of the present disclosure.

In some embodiments, the one or more amplification and/or replication steps are used for the preparation of a library to be sequenced. Any suitable amplification method may be used. Examples of amplification techniques that can be used include, but are not limited to, quantitative PCR, quantitative fluorescent PCR (QF-PCR), multiplex fluorescent PCR (MF-PCR), real time PCR (RTPCR), single cell PCR, restriction fragment length polymorphism PCR (PCR-RFLP), PCK-RFLPIRT-PCR-IRFLP, hot start PCR, nested PCR, in situ polony PCR, in situ rolling circle amplification (RCA), bridge PCR, ligation mediated PCR, Qb replicase amplification, inverse PCR, picotiter PCR and emulsion PCR. Other suitable amplification methods include the ligase chain reaction (LCR), transcription amplification, self-sustained sequence replication, selective amplification of target polynucleotide sequences, consensus sequence primed polymerase chain reaction (CP-PCR), arbitrarily primed polymerase chain reaction (AP-PCR), degenerate oligonucleotide-primed PCR (DOP-PCR) and nucleic acid-based sequence amplification (NABSA). Other amplification methods that can be used herein include those described in U.S. Pat. Nos. 5,242,794; 5,494,810; 4,988,617; and 6,582,938.

In particular embodiments, PCR is used to amplify DNA molecules after they are dispensed into individual partitions. In some cases, one or more specific priming sequences within amplification adaptors are utilized for PCR amplification. The amplification adaptors may be ligated to fragmented DNA molecules before or after dispensing into individual partitions. Polynucleotides comprising amplification adaptors with suitable priming sequences on both ends can be PCR amplified exponentially. Polynucleotides with only one suitable priming sequence due to, for example, imperfect ligation efficiency of amplification adaptors comprising priming sequences, may only undergo linear amplification. Further, polynucleotides can be eliminated from amplification, for example, PCR amplification, all together, if no adaptors comprising suitable priming sequences are ligated. In some embodiments, the number of PCR cycles vary between 10-30, but can be as low as 9, 8, 7, 6, 5, 4, 3, 2 or less or as high as 40, 45, 50, 55, 60 or more. As a result, exponentially amplifiable fragments carrying amplification adaptors with a suitable priming sequence can be present in much higher (1000-fold or more) concentration compared to linearly amplifiable or un-amplifiable fragments, after a PCR amplification. Benefits of PCR, as compared to whole genome amplification techniques (such as amplification with randomized primers or Multiple Displacement Amplification using phi29 polymerase) include, but are not limited to, a more uniform relative sequence coverage—as each fragment can be copied at most once per cycle and as the amplification is controlled by thermocycling program, a substantially lower rate of forming chimeric molecules than, for example, MDA (Lasken et al., 2007, BMC Biotechnology)—as chimeric molecules pose significant challenges for accurate sequence assembly by presenting nonbiological sequences in the assembly graph, which may result in higher rate of misassemblies or highly ambiguous and fragmented assembly, reduced sequence specific biases that may result from binding of randomized primers commonly used in MDA versus using specific priming sites with a specific sequence, a higher reproducibility in the amount of final amplified DNA product, which can be controlled by selection of the number of PCR cycles, and a higher fidelity in replication with the polymerases that are commonly used in PCR as compared to common whole genome amplification techniques.

In some embodiments, the fill-in reaction is followed by or performed as part of amplification of one or more target polynucleotides using a first primer and a second primer, wherein the first primer comprises a sequence that is hybridizable to at least a portion of the complement of one or more of the first adaptor oligonucleotides, and further wherein the second primer comprises a sequence that is hybridizable to at least a portion of the complement of one or more of the second adaptor oligonucleotides. Each of the first and second primers may be of any suitable length, such as about, less than about, or more than about 10, 15, 20, 25, 30, 35, 40, 45, 50, 55, 60, 65, 70, 75, 80, 90, 100, or more nucleotides, any portion or all of which may be complementary to the corresponding target sequence (e.g., about, less than about, or more than about 5, 10, 15, 20, 25, 30, 35, 40, 45, 50, or more nucleotides). For example, about 10 to 50 nucleotides can be complementary to the corresponding target sequence.

“Amplification” refers to any process by which the copy number of a target sequence is increased. In some cases, a replication reaction may produce only a single complementary copy/replica of a polynucleotide. Methods for primer-directed amplification of target polynucleotides include, without limitation, methods based on the polymerase chain reaction (PCR). Conditions favorable to the amplification of target sequences by PCR can be optimized at a variety of steps in the process, and depend on characteristics of elements in the reaction, such as target type, target concentration, sequence length to be amplified, sequence of the target and/or one or more primers, primer length, primer concentration, polymerase used, reaction volume, ratio of one or more elements to one or more other elements, and others, some or all of which can be altered. In general, PCR involves the steps of denaturation of the target to be amplified (if double stranded), hybridization of one or more primers to the target, and extension of the primers by a DNA polymerase, with the steps repeated (or “cycled”) in order to amplify the target sequence. Steps in this process can be optimized for various outcomes, such as to enhance yield, decrease the formation of spurious products, and/or increase or decrease specificity of primer annealing. Methods of optimization include, without limitation, adjustments to the type or number of elements in the amplification reaction and/or to the conditions of a given step in the process, such as temperature at a particular step, duration of a particular step, and/or number of cycles.

In some embodiments, an amplification reaction can comprise at least about 5, 10, 15, 20, 25, 30, 35, 40, 50, 60, 70, 80, 90, 100, 150, 200 or more cycles. In some examples, an amplification reaction can comprise at least about 20, 25, 30, 35 or 40 cycles. In some embodiments, an amplification reaction comprises no more than about 5, 10, 15, 20, 25, 35, 40, 50, 60, 70, 80, 90, 100, 150, 200 or more cycles. Cycles can contain any number of steps, such as 1, 2, 3, 4, 5, 6, 7, 8, 9, 10 or more steps. Steps can comprise any temperature or gradient of temperatures, suitable for achieving the purpose of the given step including, but not limited to, 3′ end extension (e.g., adaptor fill-in), primer annealing, primer extension, and strand denaturation. Steps can be of any duration including, but not limited to, about, less than about, or more than about 1, 5, 10, 15, 20, 25, 30, 35, 40, 45, 50, 55, 60, 70, 80, 90, 100, 120, 180, 240, 300, 360, 420, 480, 540, 600, 1200, 1800, or more seconds, including indefinitely until manually interrupted. Cycles of any number comprising different steps can be combined in any order. In some embodiments, different cycles comprising different steps are combined such that the total number of cycles in the combination is about, less that about, or more than about 5, 10, 15, 20, 25, 30, 35, 40, 50, 60, 70, 80, 90, 100, 150, 200 or more cycles. In some embodiments, amplification is performed following the fill-in reaction.

In some embodiments, the amplification reaction can be carried out on at least about 1, 2, 3, 4, 5, 6, 7, 8, 9, 10, 12, 14, 16, 18, 20, 25, 30, 40, 50, 100, 200, 300, 400, 500, 600, 800, 1000 ng of the target DNA molecule. In other embodiments, the amplification reaction can be carried out on less than about 1, 2, 3, 4, 5, 6, 7, 8, 9, 10, 12, 14, 16, 18, 20, 25, 30, 40, 50, 100, 200, 300, 400, 500, 600, 800, 1000 ng of the target DNA molecule.

Amplification can be performed before or after pooling of target polynucleotides from independent samples.

Methods of the disclosure involve determining an amount of amplifiable nucleic acid present in a sample. Any known method may be used to quantify amplifiable nucleic acid, and an exemplary method is the polymerase chain reaction (PCR), specifically quantitative polymerase chain reaction (qPCR). qPCR is a technique based on the polymerase chain reaction, and is used to amplify and simultaneously quantify a targeted nucleic acid molecule. qPCR allows for both detection and quantification (as absolute number of copies or relative amount when normalized to DNA input or additional normalizing genes) of a specific sequence in a DNA sample. The procedure follows the general principle of polymerase chain reaction, with the additional feature that the amplified DNA is quantified as it accumulates in the reaction in real time after each amplification cycle. QPCR is described, for example, in Kurnit et al. (U.S. Pat. No. 6,033,854), Wang et al. (U.S. Pat. Nos. 5,567,583 and 5,348,853), Ma et al. (The Journal of American Science, 2(3), 2006), Heid et al. (Genome Research 986-994, 1996), Sambrook and Russell (Quantitative PCR, Cold Spring Harbor Protocols, 2006), and Higuchi (U.S. Pat. Nos. 6,171,785 and 5,994,056). The contents of these are incorporated by reference herein in their entirety.

Other methods of quantification include use of fluorescent dyes that intercalate with double-stranded DNA, and modified DNA oligonucleotide probes that fluoresce when hybridized with a complementary DNA. These methods can be broadly used but are also specifically adapted to real-time PCR as described in further detail as an example. In the first method, a DNA-binding dye binds to all double-stranded (ds)DNA in PCR, resulting in fluorescence of the dye. An increase in DNA product during PCR therefore leads to an increase in fluorescence intensity and is measured at each cycle, thus allowing DNA concentrations to be quantified. The reaction is prepared similarly to a standard PCR reaction, with the addition of fluorescent (ds)DNA dye. The reaction is run in a thermocycler, and after each cycle, the levels of fluorescence are measured with a detector; the dye only fluoresces when bound to the (ds)DNA (i.e., the PCR product). With reference to a standard dilution, the (ds)DNA concentration in the PCR can be determined. Like other real-time PCR methods, the values obtained do not have absolute units associated with it. A comparison of a measured DNA/RNA sample to a standard dilution gives a fraction or ratio of the sample relative to the standard, allowing relative comparisons between different tissues or experimental conditions. To ensure accuracy in the quantification and/or expression of a target gene can be normalized with respect to a stably expressed gene. Copy numbers of unknown genes can similarly be normalized relative to genes of known copy number.

The second method uses a sequence-specific RNA or DNA-based probe to quantify only the DNA containing a probe sequence; therefore, use of the reporter probe significantly increases specificity, and allows quantification even in the presence of some non-specific DNA amplification. This allows for multiplexing, i.e., assaying for several genes in the same reaction by using specific probes with differently colored labels, provided that all genes are amplified with similar efficiency.

This method is commonly carried out with a DNA-based probe with a fluorescent reporter (e.g., 6-carboxyfluorescein) at one end and a quencher (e.g., 6-carboxy-tetramethylrhodamine) of fluorescence at the opposite end of the probe. The close proximity of the reporter to the quencher prevents detection of its fluorescence. Breakdown of the probe by the 5′ to 3′ exonuclease activity of a polymerase (e.g., Taq polymerase) breaks the reporter-quencher proximity and thus allows unquenched emission of fluorescence, which can be detected. An increase in the product targeted by the reporter probe at each PCR cycle results in a proportional increase in fluorescence due to breakdown of the probe and release of the reporter. The reaction is prepared similarly to a standard PCR reaction, and the reporter probe is added. As the reaction commences, during the annealing stage of the PCR both probe and primers anneal to the DNA target. Polymerization of a new DNA strand is initiated from the primers, and once the polymerase reaches the probe, its 5′-3′-exonuclease degrades the probe, physically separating the fluorescent reporter from the quencher, resulting in an increase in fluorescence. Fluorescence is detected and measured in a real-time PCR thermocycler, and geometric increase of fluorescence corresponding to exponential increase of the product is used to determine the threshold cycle in each reaction.

Relative concentrations of DNA present during the exponential phase of the reaction are determined by plotting fluorescence against cycle number on a logarithmic scale (so an exponentially increasing quantity will give a straight line). A threshold for detection of fluorescence above background is determined. The cycle at which the fluorescence from a sample crosses the threshold is called the cycle threshold, Ct. Since the quantity of DNA doubles every cycle during the exponential phase, relative amounts of DNA can be calculated, e.g., a sample with a Ct of 3 cycles earlier than another has 23=8 times more template. Amounts of nucleic acid (e.g., RNA or DNA) are then determined by comparing the results to a standard curve produced by a real-time PCR of serial dilutions (e.g., undiluted, 1:4, 1:16, 1:64) of a known amount of nucleic acid.

In certain embodiments, the qPCR reaction involves a dual fluorophore approach that takes advantage of fluorescence resonance energy transfer (FRET), e.g., LIGHTCYCLER hybridization probes, where two oligonucleotide probes anneal to the amplicon (see, e.g., U.S. Pat. No. 6,174,670). The oligonucleotides are designed to hybridize in a head-to-tail orientation with the fluorophores separated at a distance that is compatible with efficient energy transfer. Other examples of labeled oligonucleotides that are structured to emit a signal when bound to a nucleic acid or incorporated into an extension product include: SCORPIONS probes (e.g., Whitcombe et al., Nature Biotechnology 17:804-807, 1999, and U.S. Pat. No. 6,326,145), Sunrise (or AMPLIFLOUR) primers (e.g., Nazarenko et al., Nuc. Acids Res. 25:2516-2521, 1997, and U.S. Pat. No. 6,117,635), and LUX primers and MOLECULAR BEACONS probes (e.g., Tyagi et al., Nature Biotechnology 14:303-308, 1996 and U.S. Pat. No. 5,989,823).

In other embodiments, a qPCR reaction uses fluorescent Taqman methodology and an instrument capable of measuring fluorescence in real time (e.g., ABI Prism 7700 Sequence Detector). The Taqman reaction uses a hybridization probe labeled with two different fluorescent dyes. One dye is a reporter dye (6-carboxyfluorescein), the other is a quenching dye (6-carboxy-tetramethylrhodamine). When the probe is intact, fluorescent energy transfer occurs and the reporter dye fluorescent emission is absorbed by the quenching dye. During the extension phase of the PCR cycle, the fluorescent hybridization probe is cleaved by the 5′-3′ nucleolytic activity of the DNA polymerase. On cleavage of the probe, the reporter dye emission is no longer transferred efficiently to the quenching dye, resulting in an increase of the reporter dye fluorescent emission spectra. Any nucleic acid quantification method, including real-time methods or single-point detection methods may be used to quantify the amount of nucleic acid in the sample. The detection can be performed by several different methodologies (e.g., staining, hybridization with a labeled probe; incorporation of biotinylated primers followed by avidin-enzyme conjugate detection; incorporation of 32P-labeled deoxynucleotide triphosphates, such as dCTP or dATP, into the amplified segment), as well as any other suitable detection method for nucleic acid quantification. The quantification may or may not include an amplification step.

In some embodiments, the disclosure provides labels for identifying or quantifying the linked DNA segments. In some cases, the linked DNA segments can be labeled in order to assist in downstream applications, such as array hybridization. For example, the linked DNA segments can be labeled using random priming or nick translation.

A wide variety of labels (e.g., reporters) may be used to label the nucleotide sequences described herein including, but not limited to, during the amplification step. Suitable labels include radionuclides, enzymes, fluorescent, chemiluminescent, or chromogenic agents as well as ligands, cofactors, inhibitors, magnetic particles, and the like. Examples of such labels are included in U.S. Pat. Nos. 3,817,837; 3,850,752; 3,939,350; 3,996,345; 4,277,437; 4,275,149 and 4,366,241, which are incorporated by reference in its entirety.

Additional labels include, but are not limited to, β-galactosidase, invertase, green fluorescent protein, luciferase, chloramphenicol, acetyltransferase, β-glucuronidase, exo-glucanase and glucoamylase. Fluorescent labels may also be used, as well as fluorescent reagents specifically synthesized with particular chemical properties. A wide variety of ways to measure fluorescence are available. For example, some fluorescent labels exhibit a change in excitation or emission spectra, some exhibit resonance energy transfer where one fluorescent reporter loses fluorescence, while a second gains in fluorescence, some exhibit a loss (quenching) or appearance of fluorescence, while some report rotational movements.

Further, in order to obtain sufficient material for labeling, multiple amplifications may be pooled, instead of increasing the number of amplification cycles per reaction. Alternatively, labeled nucleotides can be incorporated in to the last cycles of the amplification reaction, e.g., 30 cycles of PCR (no label) +10 cycles of PCR (plus label).

In particular embodiments, the disclosure provides probes that can attach to the linked DNA segments. As used herein, the term “probe” refers to a molecule (e.g., an oligonucleotide, whether occurring naturally as in a purified restriction digest or produced synthetically, recombinantly or by PCR amplification), that is capable of hybridizing to another molecule of interest (e.g., another oligonucleotide). When probes are oligonucleotides, they may be single-stranded or double-stranded. Probes are useful in the detection, identification, and isolation of particular targets (e.g., gene sequences). In some cases, the probes may be associated with a label so that is detectable in any detection system including, but not limited to, enzyme (e.g., ELISA, as well as enzyme-based histochemical assays), fluorescent, radioactive, and luminescent systems

With respect to arrays and microarrays, the term “probe” is used to refer to any hybridizable material that is affixed to the array for the purpose of detecting a nucleotide sequence that has hybridized to said probe. In some cases, the probes can about 10 bp to 500 bp, about 10 bp to 250 bp, about 20 bp to 250 bp, about 20 bp to 200 bp, about 25 bp to 200 bp, about 25 bp to 100 bp, about 30 bp to 100 bp, or about 30 bp to 80 bp. In some cases, the probes can be greater than about 10 bp, about 20 bp, about 30 bp, about 40 bp, about 50 bp, about 60 bp, about 70 bp, about 80 bp, about 90 bp, about 100 bp, about 150 bp, about 200 bp, about 250 bp, about 300 bp, about 400 bp, or about 500 bp in length. For example, the probes can be about 20 to about 50 bp in length. Examples and rationale for probe design can be found in WO95/11995, EP 717,113 and WO97/29212

The probes, array of probes or set of probes can be immobilized on a support. Supports (e.g., solid supports) can be made of a variety of materials-such as glass, silica, plastic, nylon, or nitrocellulose. Supports can be rigid and have a planar surface. Supports can have from about 1 to 10,000,000 resolved loci. For example, a support can have about 10 to 10,000,000, about 10 to 5,000,000, about 100 to 5,000,000, about 100 to 4,000,000, about 1000 to 4,000,000, about 1000 to 3,000,000, about 10,000 to 3,000,000, about 10,000 to 2,000,000, about 100,000 to 2,000,000, or about 100,000 to 1,000,000 resolved loci. The density of resolved loci can be at least about 10, about 100, about 1000, about 10,000, about 100,000 or about 1,000,000 resolved loci within a square centimeter. In some cases, each resolved locus can be occupied by >95% of a single type of oligonucleotide. In other cases, each resolved locus can be occupied by pooled mixtures of probes or a set of probes. In further cases, some resolved loci are occupied by pooled mixtures of probes or a set of probes, and other resolved loci are occupied by >95% of a single type of oligonucleotide.

In some cases, the number of probes for a given nucleotide sequence on the array can be in large excess to the DNA sample to be hybridized to such array. For example, the array can have about 10, about 100, about 1000, about 10,000, about 100,000, about 1,000,000, about 10,000,000, or about 100,000,000 times the number of probes relative to the amount of DNA in the input sample.

In some cases, an array can have about 10, about 100, about 1000, about 10,000, about 100,000, about 1,000,000, about 10,000,000, about 100,000,000, or about 1,000,000,000 probes.

Arrays of probes or sets of probes may be synthesized in a step-by-step manner on a support or can be attached in presynthesized form. One method of synthesis is VLSIPS™ (as described in U.S. Pat. No. 5,143,854 and EP 476,014), which entails the use of light to direct the synthesis of oligonucleotide probes in high-density, miniaturized arrays. Algorithms for design of masks to reduce the number of synthesis cycles are described in U.S. Pat. Nos. 5,571,639 and 5,593,839. Arrays can also be synthesized in a combinatorial fashion by delivering monomers to cells of a support by mechanically constrained flowpaths, as described in EP 624,059. Arrays can also be synthesized by spotting reagents on to a support using an inkjet printer (see, for example, EP 728,520).

In some embodiments, the present disclosure provides methods for hybridizing the linked DNA segments onto an array. A “substrate” or an “array” is an intentionally created collection of nucleic acids which can be prepared either synthetically or biosynthetically and screened for biological activity in a variety of different formats (e.g., libraries of soluble molecules; and libraries of oligonucleotides tethered to resin beads, silica chips, or other solid supports). Additionally, the term “array” includes those libraries of nucleic acids which can be prepared by spotting nucleic acids of essentially any length (e.g., from 1 to about 1000 nucleotide monomers in length) onto a substrate.

Array technology and the various associated techniques and applications are described generally in numerous textbooks and documents. For example, these include Lemieux et al., 1998, Molecular Breeding 4, 277-289; Schena and Davis, Parallel Analysis with Biological Chips. in PCR Methods Manual (eds. M. Innis, D. Gelfand, J. Sninsky); Schena and Davis, 1999, Genes, Genomes and Chips. In DNA Microarrays: A Practical Approach (ed. M. Schena), Oxford University Press, Oxford, U K, 1999); The Chipping Forecast (Nature Genetics special issue; January 1999 Supplement); Mark Schena (Ed.), Microarray Biochip Technology, (Eaton Publishing Company); Cones, 2000, The Scientist 14[17]:25; Gwynn and Page, Microarray analysis: the next revolution in molecular biology, Science, 1999 Aug. 6; and Eakins and Chu, 1999, Trends in Biotechnology, 17, 217-218.

In general, any library may be arranged in an orderly manner into an array, by spatially separating the members of the library. Examples of suitable libraries for arraying include nucleic acid libraries (including DNA, cDNA, oligonucleotide, etc. libraries), peptide, polypeptide, and protein libraries, as well as libraries comprising any molecules, such as ligand libraries, among others.

The library can be fixed or immobilized onto a solid phase (e.g., a solid substrate), to limit diffusion and admixing of the members. In some cases, libraries of DNA binding ligands may be prepared. In particular, the libraries may be immobilized to a substantially planar solid phase, including membranes and non-porous substrates such as plastic and glass. Furthermore, the library can be arranged in such a way that indexing (i.e., reference or access to a particular member) is facilitated. In some examples, the members of the library can be applied as spots in a grid formation. Common assay systems may be adapted for this purpose. For example, an array may be immobilized on the surface of a microplate, either with multiple members in a well, or with a single member in each well. Furthermore, the solid substrate may be a membrane, such as a nitrocellulose or nylon membrane (for example, membranes used in blotting experiments). Alternative substrates include glass, or silica-based substrates. Thus, the library can be immobilized by any suitable method, for example, by charge interactions, or by chemical coupling to the walls or bottom of the wells, or the surface of the membrane. Other means of arranging and fixing may be used, for example, pipetting, drop-touch, piezoelectric means, inkjet and bubblejet technology, electrostatic application, etc. In the case of silicon-based chips, photolithography may be utilized to arrange and fix the libraries on the chip.

The library may be arranged by being “spotted” onto the solid substrate; this may be done by hand or by making use of robotics to deposit the members. In general, arrays may be described as macroarrays or microarrays, the difference being the size of the spots. Macroarrays can contain spot sizes of about 300 microns or larger and may be easily imaged by existing gel and blot scanners. The spot sizes in microarrays can be less than 200 microns in diameter and these arrays usually contain thousands of spots. Thus, microarrays may require specialized robotics and imaging equipment, which may need to be custom made. Instrumentation is described generally in a review by Cortese, 2000, The Scientist 14[11]:26.

Techniques for producing immobilized libraries of DNA molecules have been described. Generally, most such methods describe how to synthesize single-stranded nucleic acid molecule libraries, using, for example, masking techniques to build up various permutations of sequences at the various discrete positions on the solid substrate. U.S. Pat. No. 5,837,832 describes an improved method for producing DNA arrays immobilized to silicon substrates based on very large-scale integration technology. In particular, U.S. Pat. No. 5,837,832 describes a strategy called “tiling” to synthesize specific sets of probes at spatially defined locations on a substrate which may be used to produce the immobilized DNA libraries of the present disclosure. U.S. Pat. No. 5,837,832 also provides references for earlier techniques that may also be used. In other cases, arrays may also be built using photo deposition chemistry.

Arrays of peptides (or peptidomimetics) may also be synthesized on a surface in a manner that places each distinct library member (e.g., unique peptide sequence) at a discrete, predefined location in the array. The identity of each library member is determined by its spatial location in the array. The locations in the array where binding interactions between a predetermined molecule (e.g., a target or probe) and reactive library members occur is determined, thereby identifying the sequences of the reactive library members on the basis of spatial location. These methods are described in U.S. Pat. No. 5,143,854; WO90/15070 and WO92/10092; Fodor et al. (1991) Science, 251: 767; Dower and Fodor (1991) Ann. Rep. Med. Chem., 26: 271

To aid detection, labels can be used (as discussed above)—such as any readily detectable reporter, for example, a fluorescent, bioluminescent, phosphorescent, radioactive, etc. reporter. Such reporters, their detection, coupling to targets/probes, etc. are discussed elsewhere in this document. Labelling of probes and targets is also disclosed in Shalon et al., 1996, Genome Res 6(7):639-45.

Examples of some commercially available microarray formats are set out in Marshall and Hodgson, 1998, Nature Biotechnology, 16(1), 27-31.

In order to generate data from array-based assays a signal can be detected to signify the presence of or absence of hybridization between a probe and a nucleotide sequence. Further, direct and indirect labeling techniques can also be utilized. For example, direct labeling incorporates fluorescent dyes directly into the nucleotide sequences that hybridize to the array associated probes (e.g., dyes are incorporated into nucleotide sequence by enzymatic synthesis in the presence of labeled nucleotides or PCR primers). Direct labeling schemes can yield strong hybridization signals, for example, by using families of fluorescent dyes with similar chemical structures and characteristics, and can be simple to implement. In cases comprising direct labeling of nucleic acids, cyanine or alexa analogs can be utilized in multiple-fluor comparative array analyses. In other embodiments, indirect labeling schemes can be utilized to incorporate epitopes into the nucleic acids either prior to or after hybridization to the microarray probes. One or more staining procedures and reagents can be used to label the hybridized complex (e.g., a fluorescent molecule that binds to the epitopes, thereby providing a fluorescent signal by virtue of the conjugation of dye molecule to the epitope of the hybridized species).

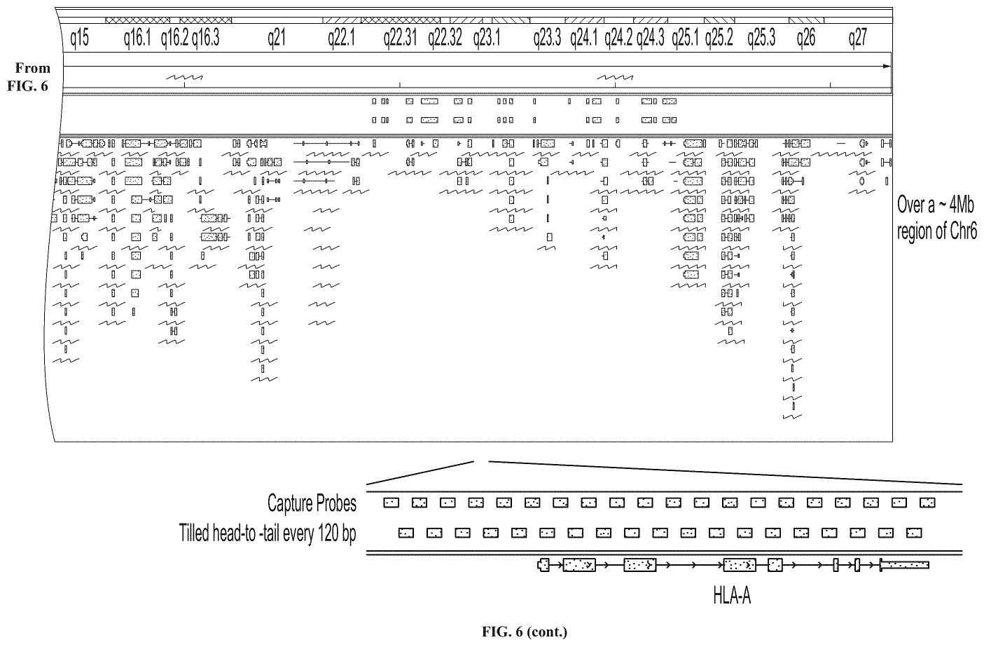

In an example, as shown in FIG. 6, enrichment for the HLA region can reduce the sequencing burden. In that example, 48 HLA loci were targeted with 2553 capture probes, each 120 bp in length. These capture probes were tiled head-to-tail every 120 bp over an approximately 4 Mb region of chromosome 6.

### Sequencing

In various embodiments, suitable sequencing methods described herein or otherwise known will be used to obtain sequence information from nucleic acid molecules within a sample. Sequencing can be accomplished through classic Sanger sequencing methods. Sequence can also be accomplished using high-throughput systems some of which allow detection of a sequenced nucleotide immediately after or upon its incorporation into a growing strand, i.e., detection of sequence in real time or substantially real time. In some cases, high-throughput sequencing generates at least 1,000, at least 5,000, at least 10,000, at least 20,000, at least 30,000, at least 40,000, at least 50,000, at least 100,000 or at least 500,000 sequence reads per hour; where the sequencing reads can be at least about 50, about 60, about 70, about 80, about 90, about 100, about 120, about 150, about 180, about 210, about 240, about 270, about 300, about 350, about 400, about 450, about 500, about 600, about 700, about 800, about 900, or about 1000 bases per read.

Sequencing can be whole-genome, with or without enrichment of particular regions of interest. Sequencing can be targeted to particular regions of the genome. Regions of the genome that can be enriched for or targeted include but are not limited to single genes (or regions thereof), gene panels, gene fusions, human leukocyte antigen (HLA) loci (e.g., Class I HLA-A, B, and C; Class II HLA-DRB1/3/4/5, HLA-DQA1, HLA-DQB1, HLA-DPA1, HLA-DPB1), exonic regions, exome, and other loci. Genomic regions can be relevant to immune response, immune repertoire, immune cell diversity, transcription (e.g., exome), cancers (e.g., BRCA1, BRCA2, panels of genes or regions thereof such as hotspot regions, somatic variants, SNVs, amplifications, fusions, tumor mutational burden (TMB), microsatellite instability (MSI)), cardiac diseases, inherited diseases, and other diseases or conditions. A variety of methods can be used to enrich for or target regions of interest, including but not limited to sequence capture. In some cases, Capture Hi-C(CHi-C) or CHi-C-like protocols are employed, employing a sequence capture step (e.g., by target enrichment array) before or after library preparation.

In some embodiments, high-throughput sequencing involves the use of technology available by Illumina's Genome Analyzer IIX, MiSeq personal sequencer, or HiSeq systems, such as those using HiSeq 2500, HiSeq 1500, HiSeq 2000, or HiSeq 1000 machines. These machines use reversible terminator-based sequencing by synthesis chemistry. These machines can do 200 billion DNA reads or more in eight days. Smaller systems may be utilized for runs within 3, 2, 1 days or less time.

In some embodiments, high-throughput sequencing involves the use of technology available by ABI Solid System. This genetic analysis platform that enables massively parallel sequencing of clonally-amplified DNA fragments linked to beads. The sequencing methodology is based on sequential ligation with dye-labeled oligonucleotides.

The next generation sequencing can comprise ion semiconductor sequencing (e.g., using technology from Life Technologies (Ion Torrent)). Ion semiconductor sequencing can take advantage of the fact that when a nucleotide is incorporated into a strand of DNA, an ion can be released. To perform ion semiconductor sequencing, a high-density array of micromachined wells can be formed. Each well can hold a single DNA template. Beneath the well can be an ion sensitive layer, and beneath the ion sensitive layer can be an ion sensor. When a nucleotide is added to a DNA, H+ can be released, which can be measured as a change in pH. The H+ ion can be converted to voltage and recorded by the semiconductor sensor. An array chip can be sequentially flooded with one nucleotide after another. No scanning, light, or cameras can be required. In some cases, an IONPROTON™ Sequencer is used to sequence nucleic acid. In some cases, an IONPGM™ Sequencer is used. The Ion Torrent Personal Genome Machine (PGM). The PGM can do 10 million reads in two hours.

In some embodiments, high-throughput sequencing involves the use of technology available by Helicos BioSciences Corporation (Cambridge, Massachusetts) such as the Single Molecule Sequencing by Synthesis (SMSS) method. SMSS is unique because it allows for sequencing the entire human genome in up to 24 hours. Finally, SMSS is described in part in US Publication Application Nos. 20060024711; 20060024678; 20060012793; 20060012784; and 20050100932.

In some embodiments, high-throughput sequencing involves the use of technology available by 454 Lifesciences, Inc. (Branford, Connecticut) such as the PicoTiterPlate device which includes a fiber optic plate that transmits chemiluminescent signal generated by the sequencing reaction to be recorded by a CCD camera in the instrument. This use of fiber optics allows for the detection of a minimum of 20 million base pairs in 4.5 hours.

Methods for using bead amplification followed by fiber optics detection are described in Marguiles, M., et al. “Genome sequencing in microfabricated high-density picolitre reactors,” Nature, doi:10.1038/nature03959; and well as in US Publication Application Nos. 20020012930; 20030068629; 20030100102; 20030148344; 20040248161; 20050079510, 20050124022; and 20060078909.

In some embodiments, high-throughput sequencing is performed using Clonal Single Molecule Array (Solexa, Inc.) or sequencing-by-synthesis (SBS) utilizing reversible terminator chemistry. These technologies are described in part in U.S. Pat. Nos. 6,969,488; 6,897,023; 6,833,246; 6,787,308; and US Publication Application Nos. 20040106110; 20030064398; 20030022207; and Constans, A., The Scientist 2003, 17(13):36.

The next generation sequencing technique can comprise real-time (SMRT™) technology by Pacific Biosciences. In SMRT, each of four DNA bases can be attached to one of four different fluorescent dyes. These dyes can be phospho linked. A single DNA polymerase can be immobilized with a single molecule of template single stranded DNA at the bottom of a zero-mode waveguide (ZMW). A ZMW can be a confinement structure which enables observation of incorporation of a single nucleotide by DNA polymerase against the background of fluorescent nucleotides that can rapidly diffuse in an out of the ZMW (in microseconds). It can take several milliseconds to incorporate a nucleotide into a growing strand. During this time, the fluorescent label can be excited and produce a fluorescent signal, and the fluorescent tag can be cleaved off. The ZMW can be illuminated from below. Attenuated light from an excitation beam can penetrate the lower 20-30 nm of each ZMW. A microscope with a detection limit of 20 zepto liters (20×10⁻²¹ liters) can be created. The tiny detection volume can provide 1000-fold improvement in the reduction of background noise. Detection of the corresponding fluorescence of the dye can indicate which base was incorporated. The process can be repeated.

In some cases, the next generation sequencing is nanopore sequencing (see, e.g., Soni G V and Meller A. (2007) Clin Chem 53: 1996-2001). A nanopore can be a small hole, of the order of about one nanometer in diameter. Immersion of a nanopore in a conducting fluid and application of a potential across it can result in a slight electrical current due to conduction of ions through the nanopore. The amount of current which flows can be sensitive to the size of the nanopore. As a DNA molecule passes through a nanopore, each nucleotide on the DNA molecule can obstruct the nanopore to a different degree. Thus, the change in the current passing through the nanopore as the DNA molecule passes through the nanopore can represent a reading of the DNA sequence. The nanopore sequencing technology can be from Oxford Nanopore Technologies; e.g., a GridlON system. A single nanopore can be inserted in a polymer membrane across the top of a microwell. Each microwell can have an electrode for individual sensing. The microwells can be fabricated into an array chip, with 100,000 or more microwells (e.g., more than 200,000, 300,000, 400,000, 500,000, 600,000, 700,000, 800,000, 900,000, or 1,000,000) per chip. An instrument (or node) can be used to analyze the chip. Data can be analyzed in real-time. One or more instruments can be operated at a time. The nanopore can be a protein nanopore, e.g., the protein alpha-hemolysin, a heptameric protein pore. The nanopore can be a solid-state nanopore made, e.g., a nanometer sized hole formed in a synthetic membrane (e.g., SiNx, or SiO2). The nanopore can be a hybrid pore (e.g., an integration of a protein pore into a solid-state membrane). The nanopore can be a nanopore with an integrated sensor (e.g., tunneling electrode detectors, capacitive detectors, or graphene-based nano-gap or edge state detectors (see e.g., Garaj et al. (2010) Nature vol. 67, doi: 10.1038/nature09379)). A nanopore can be functionalized for analyzing a specific type of molecule (e.g., DNA, RNA, or protein). Nanopore sequencing can comprise “strand sequencing” in which intact DNA polymers can be passed through a protein nanopore with sequencing in real time as the DNA translocates the pore. An enzyme can separate strands of a double stranded DNA and feed a strand through a nanopore. The DNA can have a hairpin at one end, and the system can read both strands. In some cases, nanopore sequencing is “exonuclease sequencing” in which individual nucleotides can be cleaved from a DNA strand by a processive exonuclease, and the nucleotides can be passed through a protein nanopore. The nucleotides can transiently bind to a molecule in the pore (e.g., cyclodextran). A characteristic disruption in current can be used to identify bases.

Nanopore sequencing technology from GENIA can be used. An engineered protein pore can be embedded in a lipid bilayer membrane. “Active Control” technology can be used to enable efficient nanopore-membrane assembly and control of DNA movement through the channel. In some cases, the nanopore sequencing technology is from NABsys. Genomic DNA can be fragmented into strands of average length of about 100 kb. The 100 kb fragments can be made single stranded and subsequently hybridized with a 6-mer probe. The genomic fragments with probes can be driven through a nanopore, which can create a current-versus-time tracing. The current tracing can provide the positions of the probes on each genomic fragment. The genomic fragments can be lined up to create a probe map for the genome. The process can be done in parallel for a library of probes. A genome-length probe map for each probe can be generated. Errors can be fixed with a process termed “moving window Sequencing By Hybridization (mwSBH).” In some cases, the nanopore sequencing technology is from IBM/Roche. An electron beam can be used to make a nanopore sized opening in a microchip. An electrical field can be used to pull or thread DNA through the nanopore. A DNA transistor device in the nanopore can comprise alternating nanometer sized layers of metal and dielectric. Discrete charges in the DNA backbone can get trapped by electrical fields inside the DNA nanopore. Turning off and on gate voltages can allow the DNA sequence to be read.

The next generation sequencing can comprise DNA nanoball sequencing (as performed, e.g., by Complete Genomics; see e.g., Drmanac et al. (2010) Science 327: 78-81). DNA can be isolated, fragmented, and size selected. For example, DNA can be fragmented (e.g., by sonication) to a mean length of about 500 bp. Adaptors (Adl) can be attached to the ends of the fragments. The adaptors can be used to hybridize to anchors for sequencing reactions. DNA with adaptors bound to each end can be PCR amplified. The adaptor sequences can be modified so that complementary single strand ends bind to each other forming circular DNA. The DNA can be methylated to protect it from cleavage by a type IIS restriction enzyme used in a subsequent step. An adaptor (e.g., the right adaptor) can have a restriction recognition site, and the restriction recognition site can remain non-methylated. The non-methylated restriction recognition site in the adaptor can be recognized by a restriction enzyme (e.g., Acul), and the DNA can be cleaved by Acul 13 bp to the right of the right adaptor to form linear double stranded DNA. A second round of right and left adaptors (Ad2) can be ligated onto either end of the linear DNA, and all DNA with both adaptors bound can be PCR amplified (e.g., by PCR). Ad2 sequences can be modified to allow them to bind each other and form circular DNA. The DNA can be methylated, but a restriction enzyme recognition site can remain non-methylated on the left Adl adaptor. A restriction enzyme (e.g., Acul) can be applied, and the DNA can be cleaved 13 bp to the left of the Adl to form a linear DNA fragment. A third round of right and left adaptor (Ad3) can be ligated to the right and left flank of the linear DNA, and the resulting fragment can be PCR amplified. The adaptors can be modified so that they can bind to each other and form circular DNA. A type III restriction enzyme (e.g., EcoP15) can be added; EcoP15 can cleave the DNA 26 bp to the left of Ad3 and 26 bp to the right of Ad2. This cleavage can remove a large segment of DNA and linearize the DNA once again. A fourth round of right and left adaptors (Ad4) can be ligated to the DNA, the DNA can be amplified (e.g., by PCR), and modified so that they bind each other and form the completed circular DNA template.

Rolling circle replication (e.g., using Phi 29 DNA polymerase) can be used to amplify small fragments of DNA. The four adaptor sequences can contain palindromic sequences that can hybridize, and a single strand can fold onto itself to form a DNA nanoball (DNB™) which can be approximately 200-300 nanometers in diameter on average. A DNA nanoball can be attached (e.g., by adsorption) to a microarray (sequencing flowcell). The flow cell can be a silicon wafer coated with silicon dioxide, titanium and hexamethyldisilazane (HMDS) and a photoresist material. Sequencing can be performed by unchained sequencing by ligating fluorescent probes to the DNA. The color of the fluorescence of an interrogated position can be visualized by a high-resolution camera. The identity of nucleotide sequences between adaptor sequences can be determined.

In some embodiments, high-throughput sequencing can take place using AnyDot.chips (Genovoxx, Germany). In particular, the AnyDot.chips allow for 10×-50× enhancement of nucleotide fluorescence signal detection. AnyDot.chips and methods for using them are described in part in International Publication Application Nos. WO 02088382, WO 03020968, WO 03031947, WO 2005044836, PCT/EP 05/05657, PCT/EP 05/05655; and German Patent Application Nos. DE 101 49 786, DE 102 14 395, DE 103 56 837, DE 10 2004 009 704, DE 10 2004 025 696, DE 10 2004 025 746, DE 10 2004 025 694, DE 10 2004 025 695, DE 10 2004 025 744, DE 10 2004 025 745, and DE 10 2005 012 301.

Other high-throughput sequencing systems include those disclosed in Venter, J., et al. Science 16 Feb. 2001; Adams, M. et al. Science 24 Mar. 2000; and M. J. Levene, et al. Science 299:682-686, January 2003; as well as US Publication Application No. 20030044781 and 2006/0078937. Overall such systems involve sequencing a target nucleic acid molecule having a plurality of bases by the temporal addition of bases via a polymerization reaction that is measured on a molecule of nucleic acid, i.e., the activity of a nucleic acid polymerizing enzyme on the template nucleic acid molecule to be sequenced is followed in real time. Sequence can then be deduced by identifying which base is being incorporated into the growing complementary strand of the target nucleic acid by the catalytic activity of the nucleic acid polymerizing enzyme at each step in the sequence of base additions. A polymerase on the target nucleic acid molecule complex is provided in a position suitable to move along the target nucleic acid molecule and extend the oligonucleotide primer at an active site. A plurality of labeled types of nucleotide analogs are provided proximate to the active site, with each distinguishable type of nucleotide analog being complementary to a different nucleotide in the target nucleic acid sequence. The growing nucleic acid strand is extended by using the polymerase to add a nucleotide analog to the nucleic acid strand at the active site, where the nucleotide analog being added is complementary to the nucleotide of the target nucleic acid at the active site. The nucleotide analog added to the oligonucleotide primer as a result of the polymerizing step is identified. The steps of providing labeled nucleotide analogs, polymerizing the growing nucleic acid strand, and identifying the added nucleotide analog are repeated so that the nucleic acid strand is further extended, and the sequence of the target nucleic acid is determined.

### Hi-C Methods Using Micrococcal Nuclease (MNase)

Additionally, provided herein are methods of obtaining phased HLA type information that may comprise obtaining a stabilized biological sample comprising a nucleic acid molecule complexed to at least one nucleic acid binding protein; contacting the stabilized biological sample to a micrococcal nuclease (MNase) to cleave the nucleic acid molecule into a plurality of segments; and attaching a first segment and a second segment of the plurality of segments at a junction. Use of MNase in methods herein may provide specific information about where DNA binding proteins are bound to the chromatin with up to single base pair resolution because, for example, MNase can cleave all base pairs not bound to a DNA binding protein. In addition, use of MNase digestion may allow for creation of contact maps and topologically associated domains to decipher three-dimensional chromatin structural information. In some cases, the MNase may be coupled or fused to an immunoglobulin binding protein or fragment thereof, such as a Protein A, a Protein G, a Protein A/G, or a Protein L.

For example, MNase Hi-C methods can provide locations of protein binding or genome contact interactions at a resolution of less than or equal to about 1 bp, 2 bp, 3 bp, 4 bp, 5 bp, 6 bp, 7 bp, 8 bp, 9 bp, 10 bp, 20 bp, 30 bp, 40 bp, 50 bp, 60 bp, 70 bp, 80 bp, 90 bp, 100 bp, 200 bp, 300 bp, 400 bp, 500 bp, 600 bp, 700 bp, 800 bp, 900 bp, 1000 bp, 2000 bp, 3000 bp, 4000 bp, 5000 bp, 6000 bp, 7000 bp, 8000 bp, 9000 bp, 10 kb, 20 kb, 30 kb, 40 kb, 50 kb, 60 kb, 70 kb, 80 kb, 90 kb, or 100 kb. In some cases, protein binding sites, protein footprints, contact interactions, or other features can be mapped to within 1000 bp, within 900 bp, within 800 bp, within 700 bp, within 600 bp, within 500 bp, within 400 bp, within 300 bp, within 200 bp, within 190 bp, within 180 bp, within 170 bp, within 160 bp, within 150 bp, within 140 bp, within 130 bp, within 120 bp, within 110 bp, within 100 bp, within 90 bp, within 80 bp, within 70 bp, within 60 bp, within 50 bp, within 40 bp, within 30 bp, within 20 bp, within 10 bp, within 9 bp, within 8 bp, within 7 bp, within 6 bp, within 5 bp, within 4 bp, within 3 bp, within 2 bp, or within 1 bp.

In certain aspects, methods involving a MNase digestion step may further comprise subjecting a plurality of segments to size selection to obtain a plurality of selected segments. In some cases, the plurality of selected segments can be from about 145 to about 600 bp. In some cases, the plurality of selected segments can be from about 100 to about 2500 bp. In some cases, the plurality of selected segments can be from about 100 to about 600 bp. In some cases, the plurality of selected segments can be from about 600 to about 2500 bp. In some cases, the plurality of selected segments can be from about 100 bp to about 600 bp, from about 100 bp to about 700 bp, from about 100 bp to about 800 bp, from about 100 bp to about 900 bp, from about 100 bp to about 1000 bp, from about 100 bp to about 1100 bp, from about 100 bp to about 1200 bp, from about 100 bp to about 1300 bp, from about 100 bp to about 1400 bp, from about 100 bp to about 1500 bp, from about 100 bp to about 1600 bp, from about 100 bp to about 1700 bp, from about 100 bp to about 1800 bp, from about 100 bp to about 1900 bp, from about 100 bp to about 2000 bp, from about 100 bp to about 2100 bp, from about 100 bp to about 2200 bp, from about 100 bp to about 2300 bp, from about 100 bp to about 2400 bp, or from about 100 bp to about 2500 bp.

In another aspect of methods involving a MNase digestion step as provided herein, the methods may further comprise preparing a sequencing library from the plurality of segments. In some embodiments, the method may further comprise subjecting the sequencing library to a size selection to obtain a size-selected library. In some cases, the size-selected library may be from about 350 bp to about 1000 bp in size. In some cases, the size-selected library may be from about 100 bp to about 2500 bp in size, for example, from about 100 bp to about 350 bp, from about 350 bp to about 500 bp, from about 500 bp to about 1000 bp, from about 1000 to about 1500 bp, from about 2000 bp to about 2500 bp, from about 350 bp to about 1000 bp, from about 350 bp to about 1500 bp, from about 350 bp to about 2000 bp, from about 350 bp to about 2500 bp, from about 500 bp to about 1500 bp, from about 500 bp to about 2000 bp, from about 500 bp to about 3500 bp, from about 1000 bp to about 1500 bp, from about 1000 bp to about 2000 bp, from about 1000 bp to about 2500 bp, from about 1500 bp to about 2000 bp, from about 1500 bp to about 2500 bp, or from about 2000 bp to about 2500 bp.

In another aspect, methods involving a MNase digestion step as provided herein can further comprise analyzing the plurality of segments to obtain a QC value. In some cases, a QC value may be selected from a chromatin digest efficiency (CDE) and a chromatin digest index (CDI). A CDE can be calculated as the proportion of segments having a desired length. For example, in some cases, the CDE can be calculated as the proportion of segments from 100 bp to 2500 bp in size prior to size selection. In some cases, a sample may be selected for further analysis when the CDE value is at least 65%. In some cases, a sample may be selected for further analysis when the CDE value is at least about 50%, at least about 55%, at least about 60%, at least about 65%, at least about 70%, at least about 75%, at least about 80%, at least about 85%, at least about 90%, or at least about 95%.

A CDI can be calculated as a ratio of a number of mononucleosome-sized segments to a number of dinucleosome-sized segments prior to size selection. For example, a CDI may be calculated as a logarithm of the ratio of fragments having a size of 600-2500 bp versus fragments having a size of 100-600 bp. In some cases, a sample may be selected for further analysis when the CDI value is greater than −1.5 and less than 1. In some cases, a sample may be selected for further analysis when the CDI value is greater than about −2 and less than about 1.5, greater than about −1.9 and less than about 1.5, greater than about −1.8 and less than about 1.5, greater than about −1.7 and less than about 1.5, greater than about −1.6 and less than about 1.5, greater than about −1.5 and less than about 1.5, greater than about −1.4 and less than about 1.5, greater than about −1.3 and less than about 1.5, greater than about −1.2 and less than about 1.5, greater than about −1.1 and less than about 1.5, greater than about −2 and less than about 1.5, greater than about −1 and less than about 1.5, greater than about −0.9 and less than about 1.5, greater than about −0.8 and less than about 1.5, greater than about −0.7 and less than about 1.5, greater than about −0.6 and less than about 1.5, greater than about −0.5 and less than about 1.5, greater than about −2 and less than about 1.4, greater than about −2 and less than about 1.3, greater than about −2 and less than about 1.2, greater than about −2 and less than about 1.1, greater than about −2 and less than about 1, greater than about −2 and less than about 0.9, greater than about −2 and less than about 0.8, greater than about −2 and less than about 0.7, greater than about −2 and less than about 0.6, or greater than about −2 and less than about 0.5.

In another aspect, stabilized biological samples used in methods involving a MNase digestion step as provided herein may comprise biological material that has been treated with a stabilizing agent. In some cases, the stabilized biological sample may comprise a stabilized cell lysate. Alternatively, the stabilized biological sample may comprise a stabilized intact cell. Alternatively, the stabilized biological sample may comprise a stabilized intact nucleus. In some cases, contacting the stabilized intact cell or intact nucleus sample to a MNase may be conducted prior to lysis of the intact cell or the intact nucleus. In some cases, cells and/or nuclei may be lysed prior to attaching a first segment and a second segment of a plurality of segments at a junction.

In another aspect, methods involving a MNase digestion step as provided herein may be conducted on small samples containing few cells or small amounts of nucleic acid. For example, in some cases, the stabilized biological sample may comprise fewer than 3,000,000 cells. In some cases, the stabilized biological sample may comprise fewer than 2,000,000 cells. In some cases, the stabilized biological sample may comprise fewer than 1,000,000 cells. In some cases, the stabilized biological sample may comprise fewer than 500,000 cells. In some cases, the stabilized biological sample may comprise fewer than 400,000 cells. In some cases, the stabilized biological sample may comprise fewer than 300,000 cells. In some cases, the stabilized biological sample may comprise fewer than 200,000 cells. In some cases, the stabilized biological sample may comprise fewer than 100,000 cells. In some cases, the stabilized biological sample comprises fewer than 50,000 cells. In some cases, the stabilized biological sample comprises fewer than 40,000 cells. In some cases, the stabilized biological sample comprises fewer than 30,000 cells. In some cases, the stabilized biological sample comprises fewer than 20,000 cells. In some cases, the stabilized biological sample comprises fewer than 10,000 cells. In some cases, the stabilized biological sample comprises about 10,000 cells. In some cases, the stabilized biological sample may comprise less than 10 gg DNA. In some cases, the stabilized biological sample may comprise less than 9 μg DNA. In some cases, the stabilized biological sample may comprise less than 8 μg DNA. In some cases, the stabilized biological sample may comprise less than 7 μg DNA. In some cases, the stabilized biological sample may comprise less than 6 μg DNA. In some cases, the stabilized biological sample may comprise less than 5 μg DNA. In some cases, the stabilized biological sample may comprise less than 4 μg DNA. In some cases, the stabilized biological sample may comprise less than 3 μg DNA. In some cases, the stabilized biological sample may comprise less than 2 μg DNA. In some cases, the stabilized biological sample comprises less than 1 μg DNA. In some cases, the stabilized biological sample comprises less than 0.5 μg DNA.

In another aspect, methods involving a MNase digestion step herein may be conducted on individual or single cells. For example, methods herein may be conducted on cells distributed into individual partitions. Exemplary partitions include, but are not limited to, wells, droplets in an emulsion, or surface positions (e.g., array spots, beads, etc.) comprising distinct patches of differentially sequenced linker molecules as described elsewhere herein. Additional partitions are also contemplated and consistent with the methods, compositions, and systems disclosed herein.

In additional aspects, stabilized biological samples used in methods involving a MNase digestion step herein may be further treated with an additional nuclease, such as a DNase to create fragments of DNA. In some cases, the DNase may be non-sequence specific. In some cases, the DNase may be active for both single-stranded DNA and double-stranded DNA. In some cases, the DNase may be specific for double-stranded DNA. In some cases, the DNase may preferentially cleave double-stranded DNA. In some cases, the DNase may be specific for single-stranded DNA. In some cases, the DNase may preferentially cleave single-stranded DNA. In some cases, the DNase can be DNase I. In some cases, the DNase can be DNase II. In some cases, the DNase may be selected from one or more of DNase I and DNase II. In some cases, the DNase may be coupled or fused to an immunoglobulin binding protein or fragment thereof, such as a Protein A, a Protein G, a Protein A/G, or a Protein L. Other suitable nucleases are also within the scope of this disclosure.

In additional aspects, stabilized biological samples as provided herein for use in methods involving a MNase digestion step can be treated with a crosslinking agent. In some cases, the crosslinking agent may be a chemical fixative. In some cases, the chemical fixative comprises formaldehyde, which has a spacer arm length of about 2.3-2.7 angstrom (A). In some cases, the chemical fixative comprises a crosslinking agent with a long spacer arm length, For example, the crosslinking agent can have a spacer length of at least about 3 A, 4 A, 5 A, 6 A, 7 A, 8 A, 9 A, 10 A, 11 A, 12 A, 13 A, 14 A, 15 A, 16 A, 17 A, 18 A, 19 A, or 20 A. The chemical fixative can comprise ethylene glycol bis(succinimidyl succinate) (EGS), which has a spacer arm with length about 16.1 A. The chemical fixative can comprise disuccinimidyl glutarate (DSG), which has a spacer arm with length about 7.7 A. In some cases, the chemical fixative comprises formaldehyde and EGS, formaldehyde and DSG, or formaldehyde, EGS, and DSG. In some cases where multiple chemical fixatives are employed, each chemical fixative is used sequentially; in other cases, some or all of the multiple chemical fixatives are applied to the sample at the same time. The use of crosslinkers with long spacer arms can increase the fraction of read pairs with large (e.g., >1 kb) read pair separation distances. DSG is membrane-permeable, allowing for intracellular crosslinking. DSG can increase crosslinking efficiency compared to disuccinimidyl suberate (DSS) in some applications. EGS has NHS ester reactive groups at both ends and can be reactive towards amino groups (e.g., primary amines). EGS is membrane-permeable, allowing for intracellular crosslinking. EGS crosslinks can be reversed, for example, by treatment with hydroxylamine for 3 to 6 hours at pH 8.5; in an example, lactose dehydrogenase retained 60% of its activity after reversible crosslinking with EGS. In some cases, the chemical fixative may comprise psoralen. In some cases, the crosslinking agent may be ultraviolet light, chlormethine, cyclophosphamide, chlorambucil, uramustine, melphalan, bendamustine, bis(2-chloroethyl)ethylamine, bis(2-chloroethyl)methylamine, tris(2-chloroethyl)amine, isofamide, carmustine, lomustine, streptozocin, busulfan, cisplatin, carboplatin, cicycloplatin, eptaplatin, lobaplatin, miriplatin, nedaplatin, oxaliplatin, picoplatin, satraplatin, triplatin tetranitrate, procarbazine, altretamine, dacarbazine, mitozolomide, temozolomide, mitomycin C, nitrous acid, formaldehyde, acetylaldehyde, doxorubicin, daunorubicin, epirubicin, or idarubicin. In some cases, the crosslinking agent comprises an intercalating agent, an antibiotic, or a minor groove binding agent. In some cases, the stabilized biological sample may be a crosslinked paraffin-embedded tissue sample.

In further aspects, methods involving a MNase digestion step provided herein may comprise contacting the plurality of selected segments to an antibody. In some cases, an immunoglobulin binding protein or fragment thereof tethered to an oligonucleotide adaptor may be targeted to the antibody bound to a plurality of selected segments.

In additional aspects, methods involving a MNase digestion step provided herein may comprise attaching a first segment and a second segment of a plurality of segments at a junction. In some cases, attaching may comprise filling in sticky ends using biotin tagged nucleotides and ligating the blunt ends. In some cases, attaching may comprise contacting at least the first segment and the second segment to a bridge oligonucleotide. In some cases, attaching may comprise contacting at least the first segment and the second segment to a barcode. In some embodiments, bridge oligonucleotides herein may be from at least about 5 nucleotides in length to about 50 nucleotides in length. In some embodiments, bridge oligonucleotides herein may be from about 15 to about 18 nucleotides in length. In some embodiments, bridge oligonucleotides may be about 5, about 6, about 7, about 8, about 9, about 10, about 11, about 12, about 13, about 14, about 15, about 16, about 17, about 18, about 19, about 20, about 25, about 30, about 35, about 40, about 45, or about 50 nucleotides in length. In some embodiments, bridge oligonucleotides herein may comprise a barcode.

In further aspects of methods involving a MNase digestion step herein, methods can comprise obtaining at least some sequence on each side of the junction to generate a first read pair. For example, the methods may comprise obtaining at least about 50 bp, at least about 100 bp, at least about 150 bp, at least about 200 bp, at least about 250 bp, or at least about 300 bp of sequence on each side of the junction to generate a first read pair.

In additional aspects of methods involving a MNase digestion step herein, methods can comprise mapping the first read pair to a set of contigs, and determining a path through the set of contigs that represents an order and/or orientation to a genome.

In further aspects of methods involving a MNase digestion step herein, methods can comprise mapping the first read pair to a set of contigs; and determining, from the set of contigs, a presence of a structural variant or loss of heterozygosity in the stabilized biological sample.

In additional aspects of methods involving a MNase digestion step herein, methods can comprise mapping the first read pair to a set of contigs, and assigning a variant in the set of contigs to a phase.

In further aspects of methods involving a MNase digestion step herein, methods can comprise mapping the first read pair to a set of contigs; determining, from the set of contigs, a presence of a variant in the set of contigs, and conducting a step selected from one or more of: (1) identifying a disease stage, a prognosis, or a course of treatment for the stabilized biological sample; (2) selecting a drug based on the presence of the variant; or (3) identifying a drug efficacy for the stabilized biological sample.

### Computer Systems

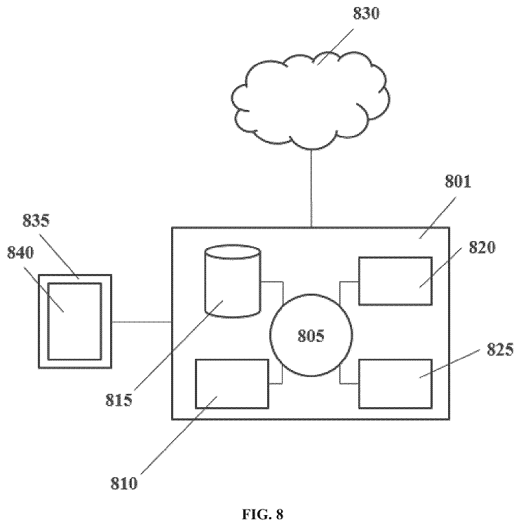

The present disclosure provides computer systems that are programmed to implement methods of the disclosure. FIG. 8 shows a computer system 801 that is programmed or otherwise configured to obtain a phased HLA type. The computer system 801 can regulate various aspects of HLA type phasing of the present disclosure, such as, for example, synthesize SNPs and indels obtained sequencing read information to produce phase information for a HLA genomic locus. The computer system 801 can be an electronic device of a user or a computer system that is remotely located with respect to the electronic device. The electronic device can be a mobile electronic device.

The computer system 801 includes a central processing unit (CPU, also “processor” and “computer processor” herein) 805, which can be a single core or multi core processor, or a plurality of processors for parallel processing. The computer system 801 also includes memory or memory location 810 (e.g., random-access memory, read-only memory, flash memory), electronic storage unit 815 (e.g., hard disk), communication interface 820 (e.g., network adapter) for communicating with one or more other systems, and peripheral devices 825, such as cache, other memory, data storage and/or electronic display adapters. The memory 810, storage unit 815, interface 820 and peripheral devices 825 are in communication with the CPU 805 through a communication bus (solid lines), such as a motherboard. The storage unit 815 can be a data storage unit (or data repository) for storing data. The computer system 801 can be operatively coupled to a computer network (“network”) 830 with the aid of the communication interface 820. The network 830 can be the Internet, an internet and/or extranet, or an intranet and/or extranet that is in communication with the Internet. The network 830 in some cases is a telecommunication and/or data network. The network 830 can include one or more computer servers, which can enable distributed computing, such as cloud computing. The network 830, in some cases with the aid of the computer system 801, can implement a peer-to-peer network, which may enable devices coupled to the computer system 801 to behave as a client or a server.

The CPU 805 can execute a sequence of machine-readable instructions, which can be embodied in a program or software. The instructions may be stored in a memory location, such as the memory 810. The instructions can be directed to the CPU 805, which can subsequently program or otherwise configure the CPU 805 to implement methods of the present disclosure. Examples of operations performed by the CPU 805 can include fetch, decode, execute, and writeback.

The CPU 805 can be part of a circuit, such as an integrated circuit. One or more other components of the system 801 can be included in the circuit. In some cases, the circuit is an application specific integrated circuit (ASIC).

The storage unit 815 can store files, such as drivers, libraries, and saved programs. The storage unit 815 can store user data, e.g., user preferences and user programs. The computer system 801 in some cases can include one or more additional data storage units that are external to the computer system 801, such as located on a remote server that is in communication with the computer system 801 through an intranet or the Internet.

The computer system 801 can communicate with one or more remote computer systems through the network 830. For instance, the computer system 801 can communicate with a remote computer system of a user (e.g., a researcher desiring phased HLA haplotypes for an individual). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 801 via the network 830.

Methods as described herein can be implemented by way of machine (e.g., computer processor) executable code stored on an electronic storage location of the computer system 801, such as, for example, on the memory 810 or electronic storage unit 815. The machine executable or machine readable code can be provided in the form of software. During use, the code can be executed by the processor 805. In some cases, the code can be retrieved from the storage unit 815 and stored on the memory 810 for ready access by the processor 805. In some situations, the electronic storage unit 815 can be precluded, and machine-executable instructions are stored on memory 810.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 801, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 801 can include or be in communication with an electronic display 835 that comprises a user interface (UI) 840 for providing, for example, synthesizing SNP and indel information obtained from sequencing reads of a HLA genomic region. Examples of UI's include, without limitation, a graphical user interface (GUI) and web-based user interface.

Methods and systems of the present disclosure can be implemented by way of one or more algorithms. One or more algorithms can be implemented by way of software upon execution by the central processing unit 805. The one or more algorithms can, for example, align a plurality of sequencing reads to a reference genome, wherein at least a portion of said plurality of sequencing reads correspond to a HLA gene locus; identify a plurality of single nucleotide polymorphisms (SNPs) and/or indels in said plurality of sequencing reads; (c) sort said plurality of SNPs and indels into phase blocks; align said plurality of sequencing reads to a variation graph to identify a plurality of HLA types; compare said plurality of HLA types to a plurality of known HLA alleles to obtain a SNP signature for each HLA allele; and (f) compare said SNP signature of (e) to said phase blocks of (c) to obtain said phased HLA type. Alternatively, the one or more algorithms can identify said plurality of SNPs by comparing or aligning said plurality of sequencing reads to said variation graph.

In some cases, the graph alignment step takes less than 30 hours, 25 hours, 20 hours, 15 hours, 10 hours, 5 hours, 4 hours, 3 hours, 2 hours, 1 hour, 50 minutes, 40 minutes, 30 minutes, 20 minutes, 10 minutes, or 5 minutes of CPU time. In some cases, the entire computational method takes less than 30 hours, 25 hours, 20 hours, 15 hours, 10 hours, 5 hours, 4 hours, 3 hours, 2 hours, 1 hour, 50 minutes, 40 minutes, 30 minutes, 20 minutes, 10 minutes, or 5 minutes of CPU time.

### Definitions

Unless defined otherwise, all technical and scientific terms used herein have the same meaning as commonly understood to one of ordinary skill in the art to which this disclosure belongs. Although any methods and reagents similar or equivalent to those described herein can be used in the practice of the disclosed methods and compositions, the exemplary methods and materials are now described.

As used herein and in the appended claims, the singular forms “a,” “and,” and “the” include plural referents unless the context clearly dictates otherwise. Thus, for example, reference to “contig” includes a plurality of such contigs and reference to “probing the physical layout of chromosomes” includes reference to one or more methods for probing the physical layout of chromosomes and equivalents thereof known to those skilled in the art, and so forth.

Also, the use of “and” means “and/or” unless stated otherwise. Similarly, “comprise,” “comprises,” “comprising,” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

The term “sequencing read” as used herein, refers to a fragment of DNA in which the sequence has been determined.

The term “subject” as used herein can refer to any eukaryotic or prokaryotic organism.

The term “read pair” or “read-pair” as used herein can refer to two or more elements that are linked to provide sequence information. In some cases, the number of read-pairs can refer to the number of mappable read-pairs. In other cases, the number of read-pairs can refer to the total number of generated read-pairs.

The term “stabilized” as used herein can describe a sample that has been preserved or otherwise protected from degradation. In some cases, a stabilized sample is crosslinked or treated with a fixative or crosslinking agent. In some cases, a stabilized sample is treated with formaldehyde, formalin, paraformaldehyde, glutaraldehyde, osmium tetroxide, or the like.

The term “about” as used herein can describe a number, unless otherwise specified, as a range of values including that number plus or minus 10% of that number.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: Phased HLA Typing

Phased HLA typing combines two independent pipelines to produce the final phased output. The first pipeline (FIG. 1, upper panel) calls and phases single nucleotide polymorphisms (SNPs) from high throughput proximity ligation reads. The second pipeline (FIG. 1, lower panel) calls HLA types from the same data but also using a database of known HLA alleles, then aligns the closest matching sequence from each called HLA allele type to the reference genome to generate a SNP signature for that allele. The SNP signature from the second process (FIG. 1, lower panel) is then matched to the high-quality phased SNPs from the first process (FIG. 1, upper panel) to determine if the SNP signature more closely matches parent 1 SNPs or parent 2 SNPs. Each allele is then assigned to the parental chromosome of the closest matching phased SNP set.

### Step 1: Align Reads to Reference

Micro-C capture reads (A) are aligned to a reference genome assembly (B, e.g., Grch38) using a read aligner, such as a BWA aligner. The output is an alignment in BAM format (C).

### Step 2: Call SNPs and Indels

SNPs are called from the alignment (C) produced in step (1). SNPs are called with any suitable software packages, for example DeepVariant (Poplin, et al. A universal SNP and small-indel variant caller using deep neural networks. Nat Biotech. 36. 2018, 983-987 which is hereby incorporated by reference in its entirety). The output of DeepVariant is a list of SNPs and small insertions and deletions (D). Using the data type, SNPs are called with high sensitivity and very high accuracy (>99%). In some cases, it is desired to use a variation graph, such as a variation graph constructed by Kourami, to call SNPs from the sequence reads. For example, in some cases alignment to a fixed reference can result in misaligned reads in homologous regions, specifically HLA-B and HLA-C, which have regions of high similarity that can sometimes result in reads from HLA-C mistakenly aligned to HLA-B, causing true SNPs not to be called.

### Step 3: Phase SNPs and Indels

SNPs and indels are sorted into phase consistent blocks using maximum-likelihood based software, such as Hapcut2 (Edge, et al. HapCUT2: reobust and accurate haplotype assembly for diverse sequencing technologies. Genome Res. 27(5). 2017, 801-812, which is hereby incorporated by reference in its entirety). The output of Hapcut2 is an assignment of SNPs to phase blocks (G). Each phase block contains a set of SNPs labeled P1 or P2 for whether they come from parental chromosome P1 or parental chromosome P2. Because the read pair sizes often vary from a few hundred bases to chromosome scale, phase blocks are obtained that span the entire 4 Mbp HLA region with over 90% of the phased SNPs in the single largest phase block. In some cases, the alleles are phased for a single gene by finding two maximum-likelihood paths through the graph. In some cases, the alleles are phased for multiple genes by finding two maximum-likelihood paths through the graph for the multiple genes.

### Step 4: Variation Graph Based Alignment

HLA allele types are called using hybrid capture proximity ligation reads. The reads are aligned to a variation graph constructed from the IPD-IMGT/HLA database of HLA variants. These alignments are examined to find the best pair of HLA types in the variation graph. Each HLA type is a tag of the form such as, for example, A*26:01:02:01 describing the major and minor HLA type groups for that allele (F). The software packages HLA*LA or Kourami are used for this purpose, though it is possible to assemble these steps independently.

### Step 5: Build HLA Type Signature Database

Each sequence in the database of sequences of known alleles (E, approximately 15,000 sequences) is aligned to the corresponding region of a reference genome assembly sequence using software. Each resulting alignment is analyzed using software to produce a list of SNPs for each called allele. This set of SNPs effectively works as a SNP signature for each allele.

### Step 6: Lookup Signature

The allele type calls from the variation graph pipeline in (4) are used to look up the SNP signatures for the sample being analyzed to produce a list of SNP signatures corresponding to the sample.

Step 7: Phase Types with SNP and Indel Matching

Each SNP in the SNP signature for an allele is compared, both sequence and location, to the phased SNPs (G) in that region. The phase, P1 or P2, with the most matching SNPs are assigned to that allele. The result of performing this for all the called alleles is a phased set of HLA types (I).

In some cases, step 4 is omitted, and HLA types are called directly by matching the allele type signature database (H) created in step 5 to the phased SNPs (G) from step 3.

In some cases, local phased assembly for each gene is performed using the capture reads. The local phased assembly of each individual gene is used in the same manner as the called type sequences (matching SNPs in the local phased assembly sequence to SNPs called with Deep Variant and phased with Hapcut2). An advantage with this approach is when the individual sample is significantly diverged from even the closest match in the database of known HLA alleles. In some cases, there is also a performance advantage since the graph alignment step requires roughly 30 wall clock hours of CPU time to compute while local phased assemblies of each gene are expected to require less time. In some cases, the graph alignment step requires roughly 30-40 minutes of CPU time to compute.

While various embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed. It is intended that the following claims define the scope of the invention and that methods and structures within the scope of these claims and their equivalents be covered thereby.

1. A method of obtaining a phased human leukocyte antigen (HLA) type of a sample comprising:
(a) aligning a plurality of sequencing reads to a reference genome or a first variation graph, wherein at least a portion of said plurality of sequencing reads correspond to an HLA gene locus;
(b) identifying a plurality of single nucleotide polymorphisms (SNPs) or indels in said plurality of sequencing reads;
(c) sorting said plurality of SNPs or indels into phase blocks;
(d) aligning said plurality of sequencing reads to a second variation graph to identify a plurality of HLA types;
(e) comparing said plurality of HLA types to a plurality of known HLA alleles to obtain a SNP signature for each HLA allele; and
(f) comparing said SNP signature of (e) to said phase blocks of (c) to obtain said phased HLA type.

2. The method of claim 1, wherein said plurality of sequencing reads are obtained by cross-linking said sample, fragmenting nucleic acids in said sample to produce nucleic acid fragments, ligating said nucleic acid fragments to produce ligated nucleic acid fragments, reversing crosslinks, and sequencing said ligated nucleic acid fragments.

3. The method of claim 2, wherein said sample is crosslinked by contacting said sample to a crosslinking agent selected from formaldehyde, psoralen, disuccinimidyl glutarate (DSG), ethylene glycol bis(succinimidyl succinate) (EGS), ultraviolet light, or a combination thereof.

4. The method of claim 2, wherein said fragmenting comprising contacting said sample to an enzyme selected from a nuclease, a restriction endonuclease, a transposase, or a combination thereof.

5. (canceled)

6. The method of claim 4, wherein said nuclease is a micrococcal nuclease.

7.-8. (canceled)

9. The method of claim 2, further comprising, subsequent to said ligating, adding a label to said nucleic acid fragments.

10. The method of claim 9, wherein said label comprises biotin, an oligonucleotide, or a combination thereof.

11. (canceled)

12. The method of claim 10, wherein said oligonucleotide comprises a barcode.

13. (canceled)

14. The method of claim 1, wherein said plurality of SNPs or indels are identified by aligning said sequencing reads to said first variation graph.

15. The method of claim 1, wherein said phased HLA type comprises HLA-A, HLA-B, HLA-C, HLA-DRB1, HLA-DRB3, HLA-DRB4, HLA-DRB5, HLA-DQA1, HLA-DQB1, HLA-DPA1, orHLA-DPB1.

16.-18. (canceled)

19. The method of claim 1, wherein the method is completed in less than 30 hours of CPU time.

20.-23. (canceled)

24. A method of obtaining a phased human leukocyte antigen (HLA) type of a sample comprising:
(a) aligning a plurality of sequencing reads to a reference genome or a variation graph, wherein at least a portion of said plurality of sequencing reads correspond to an HLA gene locus;
(b) identifying a plurality of single nucleotide polymorphisms (SNPs) or indels in said plurality of sequencing reads;
(c) sorting said plurality of SNPs and indels into a plurality of phase blocks;
(d) comparing said plurality of SNPs with a plurality of SNP signatures of known HLA types to identify a plurality of HLA types; and
(e) comparing said plurality of SNP signatures of (d) to said phase blocks of (c) to obtain said phased HLA type.

25. The method of claim 24, wherein (e) comprises assigning a phase of a HLA type of said plurality of HLA types based on a quantity of SNPs of said plurality of SNPs that match with said plurality of SNP signatures.

26. The method of claim 24, wherein (c) comprises assigning a phase to each of said plurality of phase blocks.

27. The method of claim 24, further comprising generating a database comprising a plurality of SNP signatures of known HLA types.

28. The method of claim 27, wherein said generating said database comprises aligning a plurality of HLA alleles sequences with a reference genome.

29.-30. (canceled)

31. The method of claim 24, wherein said plurality of sequencing reads comprises a contiguous sequence comprising sequences derived from regions of a chromosomes that are distal from one another in the natural sequence of the chromosome.

32. The method of claim 24, further comprising prior to a) generating a plurality of sequencing reads by (i) subjecting said sample to a proximity ligation reaction, (ii) generating a sequencing library derived from nucleic acids in said sample subjected to proximity ligation in (i), and (iii) sequencing said sequencing library.

33.-45. (canceled)

46. The method of claim 24, wherein said plurality of SNPs or indels are identified by aligning said sequencing reads to said variation graph.

47. The method of claim 24, wherein said phased HLA type comprises HLA-A, HLA-B, HLA-C, HLA-DRB1, HLA-DRB3, HLA-DRB4, HLA-DRB5, HLA-DQA1, HLA-DQB1, HLA-DPA1, orHLA-DPB1.

48.-50. (canceled)

51. The method of claim 24, wherein the method is completed in less than 30 hours of CPU time.

52.-55. (canceled)
